(19) United States
(12) Patent Application Publication
(10) Pub. No.: US 2005/0172421 A1
(43) Pub. Date: Aug. 11, 2005

(54) Keratin fibre makeup composition combining high solids content with specific rheological profile

(75) Inventors: Nathalie Jager-Lezer, Verrieres-Le-Buisson (FR); Mireille Arnaud-Roux, Aulnay Sous Bois (FR); Valerie De La Poterie, Le Chatelet En Brie (FR)

Correspondence Address: OBLON, SPIVAK, MCCLELLAND, MAIER & NEUSTADT, P.C., 1940 DUKE STREET, ALEXANDRIA, VA, 22314, US

(73) Assignee: L'OREAL, Paris (FR)

(21) Appl. No.: 10/656,378

(22) Filed: Sep. 8, 2003

Related U.S. Application Data
(60) Provisional application No. 60/417,636, filed on Oct. 11, 2002.

(30) Foreign Application Priority Data
Sep. 6, 2002 (FR) 0211092

(51) Int. Cl. A61K 7/13

(52) U.S. Cl. 8/405

(57) ABSTRACT

The invention relates to a keratin fibre makeup composition having a solids content of more than 45% of the total weight of the composition; and
a rheological profile such that, for a shear gradient range from 10⁻² to 10³ s⁻¹, the ratios of the change in shear gradient to the change in applied shear stress (the said ratio being written Δ\dot{γ}/Δτ) are not more than 7; the said composition being capable of undergoing shear stresses without breaking up over the entire breadth of the said shear gradient range. The invention allows a smooth and continuous deposition of product on the fibre.

### TECHNICAL FIELD

The present invention relates to a makeup composition for keratin fibres, particularly the eyelashes, which has a high solids content in combination with a specific flow rheology profile, with the aim of leading to a smooth and continuous deposition of product on the fibre.

### PRIOR ART

Compositions for keratin fibres, and in particular the eyelashes, may take various forms: dispersions of solid in liquid with a hydrophobic or hydrophilic continuous phase. These dispersions may take the form either of lipophilic/hydrophilic or hydrophilic/lipophilic or lipophilic/lipophilic emulsions (the 2 lipophilic phases being immiscible) or of a gel.

These compositions are characterized by a solids content which is in major part a dispersed fatty phase composed of one or more waxes in order to bring the substance onto the eyelashes and hence to bring a makeup result which is more or less volumizing (the makeup of the eyelashes is more or less thick).

Moreover, the solids of the fatty phase may also take the form of colorant (pigment, lake, nacre) and/or of pulverulent filler.

It is known from the prior art that, the higher the solids content of a composition, the greater the deposition of substance on the eyelash. Consequently, the makeup result will be volumizing.

However, increasing the solids content of the composition goes hand in hand with an increase in the consistency or viscosity at rest (under low shear, when the shear gradient \dot{γ} tends towards 0). Microscopic observation of high solids content mascaras shows poor dispersion of the solids (waxes, pigment, fillers).

Increasing the solids content is therefore limited in principle by the increase in consistency, and generally does not exceed 44% of the total weight of the composition.

Another means of enhancing the volumizing effect is to increase the adhesiveness of the product to the eyelashes by playing on the tack of the composition in order to promote the attachment of the mascara to the eyelashes and to itself in the course of successive applications of the brush. For this purpose it is possible to use additives known as tackifiers, for example, in the form of wax or of film-forming polymers.

Also in existence are mascaras referred to as volumizing mascaras, which both have a high solids content and comprise tackifying additives. Here again, however, the makeup obtained at the end is unattractive owing to the presence of lumps and the granular appearance of the deposit.

There is therefore a need for a keratin fibre makeup composition combining a high solids content with a specific rheological profile which thus allows smooth and homogeneous deposition of the said composition and which provides a makeup exhibiting a volumizing effect and which permits a rapid makeup of keratin fibres.

### DESCRIPTION OF THE INVENTION

The aim of the present invention is to provide a makeup composition for keratin fibres, especially the eyelashes which meets inter alia the abovementioned need.

The invention accordingly provides a keratin fibre makeup composition having:
a solids content of more than 45% of the total weight of the composition; and
a rheological profile such that, for a shear gradient range from 10⁻² to 10³ s⁻¹, the ratios of the change in shear gradient to the change in applied shear stress (the said ratio being written

$$\frac{Δ \dot{γ}}{Δτ} )$$

are not more than 7

$$( i . e \frac{Δ \dot{γ}}{Δτ} \leq 7 ) ,$$

the said composition being capable of undergoing shearing over the entire breadth of the said shear gradient range.

These compositions are therefore capable of undergoing shear stresses without breaking up over the entire width of the aforementioned range.

The method of measuring the solids content and the method of establishing the rheological profile are set out at the end of this description.

Preferably the said ratios are less than 5, more preferably less than 4.5 and better still less than 1.5.

Thus, by virtue of these features, compositions are obtained which exhibit a flow which is continuous (that is to say, without breaking up) and homogeneous (that is to say, without forming lumps) so as to lead to smooth and homogeneous deposition within a broad shear gradient range (from 10⁻² s−1 to 10³ s31 1).

The maximum value of the shear gradient range (10³ s−1) must be considered subject to the measurement uncertainties associated with the apparatus used. Within the range under consideration the maximum value of 10³ s−1 must be taken into account with a measurement uncertainty of ±150 s⁻¹.

Such a composition advantageously comprises a solids content at greater than 46%, preferably greater than 47%, more preferably greater than 48% or better still greater than 50% of the total weight of the composition.

In particular the composition has a solids content of less than 85%, preferably less than 75%, and better still less than 65% of the total weight of the composition.

Thus by virtue of the high overall solids content of the composition a composition is also obtained which allows a satisfactory separating and thickening effect.

In accordance with a first embodiment the aforementioned compositional features may be obtained by a composition comprising at least one fatty phase comprising at least one structuring agent of the said fatty phase.

The structurant agent may have advantageously defined properties of tack and of hardness.

The total fatty phase of the composition may represent from 10 to 60%, preferably from 15 to 50%, more preferably from 20 to 40% of the total weight of the composition.

The structurant agent may represent from from 0.1 to 60%, preferably from 5 to 55%, more preferably from 10 to 40% of the total weight of the composition.

Such a fatty-phase structuring agent may be selected from tackifying waxes, the combinations of a specific compound with at least one oil, and mixtures thereof.

The presence of one oil is particularly appropriate for a composition for treating the keratin fibres, especially the eyelashes.

Such structuring agents have the advantage of being incorporable in very large amounts which can range up to 50% by weight of the composition without substantially increasing the consistency.

It is therefore possible to incorporate these fatty-phase structuring agents into part or all of the fatty phase without greatly increasing the consistency and therefore to achieve overall solids contents which are much higher than in the prior art, with a rheological profile which conforms to the definition given above, which conveys the capacity of these compositions to be able to form a smooth, homogeneous and continuous deposit.

Another advantage is that the use of this type of compound also makes it possible to obtain compositions whose consistency allows easy and homogeneous application.

When one or more of the structuring agents is a tackifying wax, the said wax advantageously exhibits the following features:
a tack ≧0.7 N.s, in particular ranging from 0.7 to 30 N.s.; preferably ≧1 N.s, in particular ranging from 1 to 20 N.s, or better still ≧2 N.s., in particular ranging from 2 to 10 N.s; and preferentially from 2 to 5 N.s;
a hardness ≦3.5 MPa, preferably ranging from 0.01 to 3.5 MPa, more preferably still from 0.05 to 3 MPa, and even better still from 0.1 to 2.5 MPa.

The methods of measuring the tack and the hardness are set out at the end of this description.

In accordance with the invention, in the text above and below, a wax is any lipophilic, fatty compound which is solid at ambient temperature (25° C.) and atmospheric pressure (760 mmHg, or 10⁵ Pa) featuring a reversible solid/liquid change of state, having a melting point of more than 30° C. and better still more than 55° C. which can be up to 200° C., in particular up to 120° C.

By taking the wax to its melting point it is possible to make it miscible with oils and to form a microscopically homogeneous mixture, but by taking the temperature of the mixture to ambient temperature the wax recrystallizes in the oils of the mixture.

The melting point values correspond, in accordance with the invention, to the melting peak measured with the aid of a differential scanning calorimeter (DSC), for example the calorimeter sold under the name DSC 30 by Mettler, with a temperature rise of 5 or 10° C. per minute.

As tackifying wax it is possible to use a C₂₀-C₄₀ alkyl (hydroxystearyloxy)stearate (the alkyl group containing 20 to 40 carbon atoms), in particular a C₂₀-C₄₀ alkyl 12-(12′-hydroxystearyloxy)stearate, of formula (I): 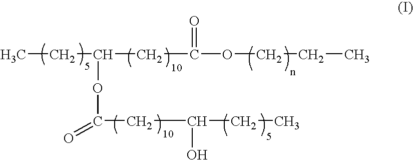
in which n is an integer ranging from 18 to 38, or a mixture of compounds of formula (I).

Such a wax is sold in particular under the names Kester Wax K 82 P and Kester Wax K 80 P by Koster Keunen.

When one or more of the structuring agents is one of the combinations mentioned above (i.e a combination of a specific compound with at least one oil), it advantageously has the following features:
a tack ≧0.1 N.s, in particular from 0.1 to 30 N.s, preferably ≧0.5 N.s, in particular from 0.5 N.s to 20 N.s, better still ≧0.8 N.s, in particular from 0.8 to 10 N.s, and even better ≧1, in particular between 1 and ;
a hardness ≦30 MPa, in particular between 0.01 to 30 MPa, preferably between 0.05 and 25 MPa, better still between 0.1 and 20 MPa.

The specific compound may be selected from semi-crystalline polymers, fatty-phase rheological agents and mixtures thereof.

Particularly, when the specific if compound is a semi-crystalline polymer, the combination of semi-crystalline polymer with at least one oil can exhibit a tack from 1 to 5 N.s and a hardness value of from 0.1 to 20 MPa.

In accordance with the invention, in the case of the abovementioned combinations, an oil is a fatty substance which is liquid at ambient temperature.

Moreover, a volatile compound, for example a volatile oil, is, for the purposes of the invention, any compound (or non-aqueous medium) capable of undergoing evaporation on contact with the skin or with the keratin fibre in less than one hour at ambient temperature and atmospheric pressure. The volatile compound is a volatile cosmetic compound which is liquid at ambient temperature and has in particular a non-zero vapour pressure at ambient temperature and atmospheric pressure, in particular has a vapour pressure ranging from 0.13 Pa to 40 000 Pa (10⁻³ to 300 mmHg), in particular ranging from 1.3 Pa to 13 000 Pa (0.01 to 100 mmHg), and more particularly ranging from 1.3 Pa to 1 300 Pa (0.01 to 10 mmHg).

Conversely, a non-volatile compound, for example non-volatile oil, is a compound remaining on the skin or keratin fibre at ambient temperature and atmospheric pressure for at least a number of hours and having in particular a vapour pressure of less than 10⁻³ mmHg (0.13 Pa).

The oil may be selected from all physiologically acceptable oils and in particular cosmetically acceptable oils, especially mineral oils, animal oils, vegetable oils, synthetic oils; in particular volatile or non-volatile hydrocarbon oils and/or silicone oils and/or fluoro oils and mixtures thereof.

More specifically a hydrocarbon oil is an oil comprising principally atoms of carbon and hydrogen and optionally one or more functions selected from hydroxyl, ester, ether and carboxyl functions. Generally the oil exhibits a viscosity of from 0.5 to 100 000 cps, preferably from 50 to 50 000 cps and more preferably from 100 to 300 000 cps.

Possible examples of oils which can be used in the invention include
hydrocarbon oils of animal origin such as perhydrosqualene;
vegetable hydrocarbon oils such as liquid triglycerides of fatty acids of 4 to 24 carbon atoms, such as the triglycerides of heptanoic or octanoic acid or else sunflower oil, maize oil, soya oil, marrow oil, grapeseed oil, sesame oil, hazelnut oil, apricot oil, macadamia oil, castor oil or avocado oil, triglycerides of caprylic/capric acids such as those sold by Stearineries Dubois or those sold under the names Miglyol 810, 812 and 818 by Dynamit Nobel, jojoba oil and karite butter;
linear or branched hydrocarbons of mineral or synthetic origin, such as liquid paraffins and their derivatives, vaseline, polydecenes, polybutenes, and hydrogenated polyisobutene such as Parleam;
synthetic esters and ethers in particular of fatty acids, such as the oils of formula R₁COOR₂ in which R₁ represents the residue of a higher fatty acid containing 1 to 40 carbon atoms and R₂ represents a hydrocarbon chain containing 1 to 40 carbon atoms, with R₁+R₂≧10, such as, for example, Purcellin oil, isononyl isononanoate, isopropyl myristate, 2-ethylhexyl palmitate, 2-octyldodecyl stearate, 2-octyldodecyl erucate, isostearyl isostearate and tridecyl trimellitate; hydroxylated esters such as isostearyl lactate, octyl hydroxystearate, octyldodecyl hydroxystearate, diisostearyl malate, triisocetyl citrate, and heptanoates, octanoates and decanoates of fatty alcohols; polyol esters such as propylene glycol dioctanoate, neopentylglycol diheptanoate, and diethylene glycol diisononanoate; and pentaerythritol esters such as pentaerythrityl tetraisostearate;
fatty alcohols having 12 to 26 carbon atoms, such as octyldodecanol, 2-butyloctanol, 2-hexyldecanol, 2-undecylpentadecanol and oleyl alcohol;
fluoro oils, optionally with a partial hydrocarbon and/or silicone content;
silicone oils such as linear or cyclic, volatile or nonvolatile polydimethylsiloxanes (PDMS); polydimethylsiloxanes containing alkyl, alkoxy or phenyl groups, pendant or at the silicone chain end, groups having 2 to 24 carbon atoms; phenylated silicones such as phenyltrimethicones, phenyl-dimethicones, phenyltrimethylsiloxydiphenylsiloxanes, diphenyldimethicones, diphenylmethyldiphenyl-trisiloxanes and 2-phenylethyl trimethylsiloxy-silicates,
and mixtures thereof.

The oil preferably has a molecular mass of greater than or equal to 250 g/mol, in particular between 250 and 10 000 g/mol, preferably greater than or equal to 300 g/mol, in particular between 300 and 8 000 g/mol, and better still greater than or equal to 400 g/mol, in particular between 400 and 5 000 g/mol. This oil may be selected from
polybutylenes such as Indopol H-100 (of molar mass or MM=965 g/mol), Indopol H-300 (MM=1 340 g/mol) and Indopol H-1500 (MM=2 160 g/mol), which are sold or manufactured by Amoco;
hydrogenated polyisobutylenes such as Panalane H-300 E, sold or manufactured by Amoco (M=1 340 g/mol), Viseal 20 000, sold or manufactured by Synteal (MM=6 000 g/mol) and Rewopal PIB 1 000, sold or manufactured by Witco (MM=1 000 g/mol);
polydecenes and hydrogenated polydecenes such as Puresyn 10 (MM=723 g/mol) and Puresyn 150 (MM=9 200 g/mol), sold or manufactured by Mobil Chemicals;
esters such as
linear fatty acid esters having a total carbon number ranging from 30 to 70, such as pentaerythrityl tetrapelargonate (MM=697.05 g/mol);
hydroxylated esters such as diisostearyl malate (MM=639 g/mol),
aromatic esters such as tridecyl trimellitate (MM=757.19 g/mol),
C₂₄-C₂₈ branched fatty acid or fatty alcohol esters, such as those described in EP-A-0 955 039, and in particular triisocetyl citrate (MM=865 g/mol), pentaerythrityl tetraisononanoate (MM=697.05 g/mol), glyceryl triisostearate (MM=891.51 g/mol), glyceryl 2-tridecyltetradecanoate (MM=1 143.98 g/mol), pentaerythrityl tetraisostearate (MM=1 202.02 g/mol), poly-2-glyceryl tetraisostearate (MM=1 232.04 g/mol) or else pentaerythrityl 2-tetradecyltetradecanoate (MM=1 538.66 g/mol),
oils of plant origin such as sesame oil (820.6 g/mol), and mixtures thereof.

In accordance with the invention, in the case of the abovementioned combinations, the term “polymer” refers to compounds containing at least two repeating units, preferably at least 3 repeating units and more especially at least 10 repeating units. The term “semi-crystalline polymer” refers to polymers containing a crystallizable portion, a crystallizable pendant chain or a crystallizable sequence in the skeleton, and an amorphous portion in the skeleton and having a first-order reversible phase-change temperature, in particular of fusion (solid-liquid transition). When the crystallizable portion is in the form of a crystallizable sequence of the polymeric skeleton, the amorphous portion of the polymer is in the form of an amorphous sequence; in this case the semi-crystalline polymer is a block copolymer of, for example, diblock, triblock or multiblock type comprising at least one crystallizable block and at least one amorphous block. A block generally comprises at least 5 identical repeating units. The crystallizable block or blocks are then different in chemical nature from the amorphous block or blocks.

The semi-crystalline polymer according to the invention has a melting point of greater than or equal to 30° C. (in particular ranging from 30° C. to 80° C.), preferably ranging from 30° C. to 60° C. This melting point is a first-order change-of-state temperature.

This melting point can be measured by any known method and in particular by means of a differential scanning calorimeter (DSC).

Advantageously the semi-crystalline polymer or polymers to which the invention applies exhibit a number-average molecular mass of greater than or equal to 1 000.

Advantageously the semi-crystalline polymer or polymers of the composition of the invention have a number-average molecular mass \overline{M}n ranging from 2 000 to 800 000, preferably from 3 000 to 500 000, better still from 4 000 to 150 000, and in particular less than 100 000, and better still from 4 000 to 99 000. Preferably they have a number-average molecular mass of more than 5 600, ranging for example from 5 700 to 99 000.

A crystallizable chain or block in the sense of the invention is a chain or block which if it were alone would pass reversibly from the amorphous state to the crystalline state depending on whether the temperature was above or below the melting point. A chain in the sense of the invention is a group of atoms which is pendant or lateral relative to the skeleton of the polymer. A block is a group of atoms belonging to the skeleton, the group constituting one of the repeating units of the polymer. Advantageously the “crystallizable pendant chain” may be a chain containing at least 6 carbon atoms.

The crystallizable block(s) or chain(s) of the semi-crystalline polymers preferably represent at least 30% of the total weight of each polymer and better still at least 40%. The semi-crystalline polymers of the invention containing crystallizable blocks are block or multiblock polymers. They can be obtained by polymerizing monomers containing reactive double bonds (or ethylenic bonds) or by polycondensation. When the polymers of the invention are polymers containing crystallizable side chains they are advantageously in random or statistical form.

The semicrystalline polymers of the invention are preferably synthetic in origin. Moreover, they do not include a polysaccharide skeleton. Generally speaking, the crystallizable units (chains or blocks) of the semi-crystalline polymers according to the invention originate from one or more monomers containing crystallizable block(s) or chain(s) used for the preparation of the semi-crystalline polymers.

In accordance with the invention, the semi-crystalline polymer can be selected from block copolymers containing at least one crystallizable block and at least one amorphous block, homopolymers and copolymers carrying at least one crystallizable side chain per repeating unit, and mixtures thereof.

The semi-crystalline polymers which can be used in the invention are in particular:
block polyolefin copolymers of controlled crystallization, particularly those whose monomers are described in EP-A-0 951 897,
polycondensates, particularly those of aliphatic or aromatic, polyester type or aliphatic/aromatic copolyester type,
homopolymers or copolymers carrying at least one crystallizable side chain and homopolymers or copolymers carrying in the skeleton at least one crystallizable block, such as those described in U.S. Pat. No. 5,156,911,
homopolymers or copolymers carrying at least one crystallizable side chain, in particular containing one or more fluorine-containing groups, such as those described in WO-A-01/19333,
and mixtures thereof. In these two latter cases the crystallizable side chain(s) or block(s) are hydrophobic.
   A) Semi-Crystalline Polymers having Crystallizable Side Chains

Mention may be made in particular of those defined in U.S. Pat. No. 5,156,911 and WO-A-01/19333. These are homopolymers or copolymers containing from 50 to 100% by weight of units resulting from the polymerization of one or more monomers which carry a crystallizable hydrophobic side chain.
These homopolymers or copolymers are of any kind, provided that they meet the conditions indicated above.

They may result
from the polymerization, in particular the free-radical polymerization, of one or more monomers containing reactive or ethylenic double bond(s) in respect of a polymerization, namely containing a vinylic, (meth)acrylic or allylic group;
from the polycondensation of one or more monomers which carry co-reactive groups (carboxylic or sulphonic acid, alcohol, amine or isocyanate), such as, for example, polyesters, polyurethanes, polyethers, polyureas and polyamides.

Generally speaking these polymers are selected in particular from homopolymers and copolymers resulting from the polymerization of at least one crystallizable-chain monomer which can be represented by formula X: 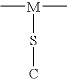
with M representing an atom of the polymeric skeleton, S representing a spacer and C representing a crystal-lizable group.

The crystallizable chains “—S—C” may be aliphatic or aromatic, optionally fluorinated or perfluorinated. “S” represents in particular a (CH₂)n or (CH₂CH₂O)n or (CH₂O) group which is linear or branched or cyclic, with n being an integer ranging from 0 to 22. “S” is preferably a linear group. Preferably “S” and “C” are different.

When the crystallizable chains “—S—C” are aliphatic hydrocarbon chains, they contain alkyl hydrocarbon chains containing at least 11 carbon atoms and not more than 40 carbon atoms, and better still not more than 24 carbon atoms. They are in particular aliphatic chains or alkyl chains possessing at least 12 carbon atoms and preferably the chains in question are C₁₄-C₂₄ alkyl chains. When the chains in question are fluorinated or perfluorinated alkyl chains they contain at least 6 fluorinated carbon atoms and in particular at least 11 carbon atoms of which at least 6 carbon atoms are fluorinated.

Examples of semi-crystalline polymers or copolymers containing one or more crystallizable chains include those resulting from the polymerization of one or more monomers as follows: saturated alkyl (meth)acrylates with the alkyl group C₁₄-C₂₄, perfluoro-alkyl (meth)acrylates with a C₁₁-C₁₅ perfluoroalkyl group, N-alkyl(meth)acrylamides with a C₁₄ to C₂₄ alkyl group with or without a fluorine atom, vinyl esters containing alkyl or perfluoro(alkyl) chains with a C₁₄ to C₂₄ alkyl group (with at least 6 fluorine atoms for a perfluoroalkyl chain), vinyl ethers containing alkyl or perfluoro(alkyl) chains with a C₁₄ to C₂₄ alkyl group and at least 6 fluorine atoms for a perfluoroalkyl chain, C₁₄ to C₂₄ alpha-olefins such as, for example, octadecene, para-alkylstyrenes with an alkyl group containing 12 to 24 carbon atoms, and mixtures thereof.

When the polymers result from a polycondensation, the hydrocarbon and/or fluorinated crystallizable chains as defined above are carried by a monomer which may be a diacid, a diol, a diamine or a diisocyanate.

When the polymers of the invention are copolymers, they also include from 0 to 50% of Y or Z groups resulting from the copolymerization

α) of Y, which is a polar or non-polar monomer or a mixture of the two:

When Y is a polar monomer, it is either a monomer which carries polyoxyalkylenated groups (especially oxyethylenated and/or oxypropylenated), a hydroxyalkyl (meth)acrylate such as hydroxyethyl acrylate, (meth)acrylamide, and N-alkyl(meth)-acrylamide, and N,N-dialkyl(meth)acrylamide such as, for example, N,N-diisopropylacrylamide or N-vinyl-pyrrolidone (NVP), N-vinylcaprolactam, a monomer which carries at least one carboxylic acid group such as (meth)acrylic, crotonic, itaconic, maleic or fumaric acid or which carries a carboxylic anhydride group such as maleic anhydride, and mixtures thereof.
When Y is a non-polar monomer it may be an ester of the linear, branched or cyclic alkyl (meth)acrylate type, a vinyl ester, an alkyl vinyl ether, an alpha-olefin, styrene or styrene substituted by a C₁ to C₁₀ alkyl group, such as α-methylstyrene or a polyorganosiloxane-type macromonomer containing vinylic unsaturation.

“Alkyl” for the purposes of the invention is a saturated group, in particular a C₈ to C₂₄ group, unless specifically mentioned, and better still a C₁₄ to C₂₄ group.
β) of Z which is a polar monomer or a mixture of polar monomers. In this case Z has the same definition as “polar Y” defined above.

Semi-crystalline polymers containing a crystallizable side chain are preferably alkyl (meth) acrylate or alkyl (meth) acrylamide homopolymers with an alkyl group as defined above, and in particular C₁₄-C₂₄ group, copolymers of these monomers with a hydrophilic monomer, preferably of a different kind from (meth)acrylic acid, such as N-vinylpyrrolidone, or hydroxyethyl (meth)acrylate, and mixtures thereof.

B) Polymers Carrying in the Skeleton at Least One Crystallizable Block

These polymers are, in particular, block copolymers consisting of at least 2 chemically different blocks of which one is crystallizable.
It is possible to use the block polymers defined in patent U.S. Pat. No. 5,156,911;
block copolymers of olefin or of cycloolefin containing a crystallizable chain, such as those obtained from the block polymerization of
   cyclobutene, cylohexene, cyclooctene, norbornene (i.e. bicyclo[2.2.1]hept-2-ene), 5-methyl-norbornene, 5-ethylnorbornene, 5,6-dimethylnorbornene, 5,5,6-trimethylnorbornene, 5-ethylidenenorbornene, 5-phenylnorbornene, 5-benzylnorbornene, 5-vinylnor-bornene, 1,4,5,8-dimethano-1,2,3,4,4a,5,8a-octahydro-naphthalene, dicyclopentadiene or mixtures thereof
   with ethylene, propylene, 1-butene, 3-methyl-l-butene, 1-hexene, 4-methyl-1-pentene, 1-octene, 1-decene, 1-eicosene or mixtures thereof,
   and in particular block copoly(ethylene/norbornene) and block (ethylene/propylene/ethylidene-norbornene) terpolymers. It is also possible to use those resulting from the block copolymerization of at least two C₂-C₁₆ and better still C₂-C₁₂ and even better still C₄-C₁₂ α-olefins, such as those mentioned above, and in particular the block bipolymers of ethylene and of 1-octene.
The copolymers may be copolymers having at least one crystallizable block, the remainder of the copolymer being amorphous (at ambient temperature). These copolymers may further comprise two chemically different crystallizable blocks. Preferred copolymers are those which possess at ambient temperature both a crystallizable block and an amorphous block which is both hydrophobic and lipophilic, the blocks being distributed sequentially; mention may be made, for example, of polymers possessing one of the following crystallizable blocks and one of the following amorphous blocks:
   Naturally crystallizable blocks, a) polyesters such as poly(alkylene terephthalate), b) polyolefins such as polyethylenes or polypropylenes.
   Amorphous and lipophilic block, such as amorphous polyolefins or copoly(olefin)s such as poly(isobutylene), hydrogenated polybutadiene and hydrogenated poly(isoprene).

Possible examples of such copolymers containing a crystallizable block and an amorphous block, the blocks being distinct, include:
α) block poly(ε-caprolactone)-b-poly(butadiene) copolymers, used preferably in hydrogenated form, such as those described in the article “Melting behavior of poly(ε-caprolactone)-block-polybutadiene copolymers” by S. Nojima, Macromolecules, 32, 3727-3734 (1999).
β) hydrogenated poly(butylene terephthalate)-b-poly(isoprene) block or multiblock copolymers, cited in the article “Study of morphological and mechanical properties of PP/PBT” by B. Boutevin et al., Polymer Bulletin, 34, 117-123 (1995).
γ) the poly(ethylene)-b-copoly(ethylene/-propylene) block copolymers cited in the articles “Morphology of semi-crystalline block copolymers of ethylene-(ethylene-alt-propylene)” by P. Rangarajan et al., Macromolecules, 26, 4640-4645 (1993) and “Polymer aggregates with crystalline cores: the system poly(ethylene)-poly(ethylene-propylene)” by P. Richter et al., Macromolécules, 30, 1053-1068 (1997).
δ) the poly(ethylene)-b-poly(ethylethylene) block copolymers cited in the general article “Crystallization in block copolymers” by I. W. Hamley, Advances in Polymer Science, Vol. 148, 113-137 (1999).

The semi-crystalline polymers of the composition of the invention may be non-crosslinked or partially crosslinked, provided that the degree of crosslinking is not detrimental to their dissolution or dispersion in the liquid fatty phase by heating above their melting point. This may then be a chemical crosslinking, by reaction with a polyfunctional monomer during the polymerization. It may also be a physical crosslinking, which may then be due either to the establishment of bonds of hydrogen type or dipolar type between groups carried by the polymer, such as, for example, the dipolar interactions between carboxylate ionomers, these interactions being low in quantity and borne by the polymer skeleton; or due to phase separation between the crystallizable blocks and the amorphous blocks carried by the polymer.

The semi-crystalline polymers of the composition according to the invention are preferably non-crosslinked.

According to one particular embodiment of the invention, the polymer is selected from copolymers resulting from the polymerization of at least one monomer containing a crystallizable chain, selected from saturated C₁₄ to C₂₄ alkyl (meth)acrylates, C₁₁ to C₁₅ perfluoroalkyl (meth)acrylates, C₁₄ to C₂₄ N-alkyl-(meth)acrylamides with or without a fluorine atom, vinyl esters containing C₁₄ to C₂₄ alkyl or perfluoroalkyl chains, vinyl ethers containing C₁₄ to C₂₄ alkyl or perfluoroalkyl chains, C₁₄ to C₂₄ alpha-olefins, para-alkylstyrenes with an alkyl group containing 12 to 24 carbon atoms, with at least one optionally fluorinated C₁ to C₁₀ monocarboxylic ester or amide, which can be represented by the following formula: 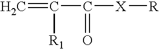
in which R₁ is H or CH₃, R represents an optionally fluorinated C₁-C₁₀ alkyl group and X represents O, NH or NR₂, where R₂ represents an optionally fluorinated C₁-C₁₀ alkyl group.

According to one more particular embodiment of the invention the polymer is derived from a monomer containing a crystallizable chain, selected from saturated C₁₄ to C₂₂ alkyl (meth)acrylates.

As a specific example of a structuring semi-crystalline polymer that can be used in the composition according to the invention, mention may be made of the Interlimer® products from Landec which are described in the brochure “Interlimer® polymers”, Landex IP22 (Rev. 4-97). These polymers are in solid form at ambient temperature (25° C.). They carry crystallizable side chains and have the above formula X.

The semi-crystalline polymers may in particular be: those described in Examples 3, 4, 5, 7, 9 and 13 of patent U.S. Pat. No. 5,156,911 containing a —COOH group, resulting from the copolymerization of acrylic acid and C₅ to C₁₆ alkyl (meth)acrylate, and more particularly from the copolymerization:
of acrylic acid, hexadecyl acrylate and isodecyl acrylate in a 1/16/3 weight ratio,
of acrylic acid and pentadecyl acrylate in a 1/19 weight ratio,
of acrylic acid, hexadecyl acrylate and ethyl acrylate in a 2.5/76.5/20 weight ratio,
of acrylic acid, hexadecyl acrylate and methyl acrylate in a 5/85/10 weight ratio,
of acrylic acid and octadecyl methacrylate in a 2.5/97.5 weight ratio,
of hexadecyl acrylate, polyethylene glycol methacrylate monomethyl ether containing 8 ethylene glycol units, and acrylic acid in an 8.5/1/0.5 weight ratio.

It is also possible to use the structure “O” from National Starch, as described in U.S. Pat. No. 5,736,125, with a melting point of 44° C., and also semi-crystalline polymers containing crystallizable pendant chains containing fluorinated groups, as described in Examples 1, 4, 6, 7 and 8 of WO-A-01/19333.

It is additionally possible to use the semi-crystalline polymers obtained by copolymerizing stearyl acrylate and acrylic acid or NVP, as described in U.S. Pat. No. 5,519,063 or EP-A-550745, with melting points of 40° C. and 38° C. respectively.

It is also possible to use semi-crystalline polymers obtained by copolymerizing behenyl acrylate and acrylic acid or NVP, as described in U.S. Pat. No. 5,519,063 and EP-A-550745 with melting points of 60° C. and 58° C. respectively.

The semi-crystalline polymers preferably do not contain a carboxyl group.

The other abovementioned combinations may comprise the combination of a rheological agent with at least one oil.

This rheological agent is capable of thickening and/or gelling the oil phase. It may be present in an amount which is effective for increasing the viscosity of this phase, particularly until a solid gel is obtained, namely a product which does not flow under its own weight.

The rheological agent is advantageously selected from lipophilic gelling agents, organic gellers and mixtures thereof.

The lipophilic gelling agent may be organic or mineral, polymeric or molecular.

As inorganic lipophilic gelling agent mention may be made of optionally modified clays, such as hectorites modified with a C₁₀ to C₂₂ fatty acid ammonium chloride, such as hectorite modified with distearyldimethylammonium chloride.

Mention may also be made of optionally surface-hydrophobicized pyrogenic silica whose particle size is less than 1 μm. It is possible in effect to modify chemically the surface of the silica, by a chemical reaction which brings about a decrease in the number of silanol groups present on the surface of the silica. In particular it is possible to substitute the silanol groups by hydrophobic groups: in that case a hydrophobic silica is obtained. The hydrophobic groups may be
trimethylsiloxyl groups, obtained in particular by treating pyrogenic silica in the presence of hexamethyldisilazane. Silicas treated in this way are named “silica silylate” according to the CTFA (6th edition, 1995). They are sold, for example, under the names Aerosil R812® by Degussa and CAB—O—SIL TS-530® by Cabot.
dimethylsilyloxyl or polydimethylsiloxane groups, obtained in particular by treating pyrogenic silica in the presence of polydimethylsiloxane or dimethydichlorosilane. Silicas treated in this way are named “silica dimethyl silylate” according to the CTFA (6th edition, 1995). They are sold for example under the names Aerosil R972®, Aerosil R974® by Degussa and CAB-O-SIL TS-610® and CAB-O-SIL TS-720® by Cabot.

The hydrophobic pyrogenic silica preferably has a particle size which can be nanometric to micrometric, ranging for example from 5 to 200 nm approximately.

The polymeric organic lipophilic gelling agents are, for example, partly or completely crosslinked elastomeric organopolysiloxanes of three-dimensional structure, such as those sold under the names KSG6, KSG16 and KSG18 by Shin-Etsu, Trefil E-505C or Trefil E-506C by Dow-Corning, Gransil SR-CYC, SR DMF10, SR-DC556, SR 5CYC gel, SR DMF 10 gel, SR DC 556 gel from Grant Industries, SF 1204 and JK 113 by General Electric; ethylcellulose such as those sold under the name Ethocel by Dow Chemical; polyamides. such as copolymers of a C₃₆ diacid condensed with ethylene-diamine, with a number-average molecular mass of approximately 6000, such as the compounds sold by Arizona Chemical under the names Uniclear 80 and Uniclear 100, gums, especially silicone gums, such as PDMS having a viscosity >100 000 centistokes, galacto-mannans containing from one to six and better still from two to four hydroxyl groups per saccharide unit, substituted by a saturated or unsaturated alkyl chain, such as guar gum alkylated with C₁ to C₆ alkyl chains and better still C₁ to C₃ alkyl chains, and mixtures thereof.

As preferred lipophilic gelling agent, use is made of non-polymeric molecular organic gelling agents, also dubbed organic gellers, which are compounds whose molecules are capable of establishing, between them-selves, physical interactions leading to self-aggregation of the molecules with formation of a 3D macromolecular network which is responsible for the gelation of the liquid fatty phase.

A liquid fatty phase in the sense of the invention is a fatty phase which is liquid at ambient temperature (25° C.) and atmospheric pressure (760 mmHg or 105 Pa) and is composed of one or more fatty substances which are liquid at ambient temperature, also called oils, which are generally mutually compatible.

The macromolecular network may result from the formation of a network of fibrils (owing to stacking or aggregation of organic geller molecules), which immobilizes the molecules of the liquid fatty phase.

The ability to form this network of fibrils, and hence to gel, depends on the nature (or chemical class) of the organic geller, on the nature of the substituents carried by its molecules for a given chemical class, and on the nature of the liquid fatty phase.

The physical interactions are diverse but exclude co-crystallization. These physical interactions are, in particular, interactions such as self-complementary hydrogen interactions, π interactions between unsaturated rings, dipolar interactions, coordination bonds with organometallic derivatives, and combinations thereof. In general, each molecule of an organic geller can establish a number of types of physical interaction with a neighbouring molecule. Thus, advantageously, the molecules of the organic gellers according to the invention include at least one group capable of establishing hydrogen bonds and, better still, at least two groups capable of establishing hydrogen bonds, at least one aromatic ring, and better still at least two aromatic rings, at least one or more bonds with ethylenic unsaturation and/or at least one or more asymmetric carbons. The groups capable of forming hydrogen bonds are preferably selected from hydroxyl, carbonyl, amine, carboxylic acid, amide, urea and benzyl groups and combinations thereof.

The organic geller or gellers according to the invention are soluble in the liquid fatty phase after heating to give a transparent homogeneous liquid phase. They may be solid or liquid at ambient temperature and atmospheric pressure.

The molecular organic geller or gellers which can be used in the composition according to the invention are, in particular, those described in the document “Specialist Surfactants”, edited by D. Robb, 1997, p. 209-263, Chapter 8 by P. Terech, European applications EP-A-1068854 and EP-A-1086945 or else application WO-A-02/47031.

Among these organic gellers mention may be made in particular of the amides of carboxylic acids, especially tricarboxylic acids, such as cyclohexane-tricarboxamides (see European patent application EP-A-1068854), diamides having hydrocarbon chains each containing 1 to 22 carbon atoms, for example 6 to 18 carbon atoms, the said chains being unsubstituted or substituted by at least one substituent selected from ester, urea and fluoro groups (see application EP-A-1086945), and in particular the diamides resulting from the reaction of diaminocyclohexane, in particular diaminocyclohexane in trans form, and an acid chloride such as, for example, N,N′-bis(dodecanoyl)-1,2-diamino-cyclohexane, the amides of N-acylamino acids such as the diamides resulting from the action of an N-acylamino acid with amines containing 1 to 22 carbon atoms, such as, for example, those described in WO-93/23008, and . especially the amides of N-acylglutamic acid in which the acyl group represents a C₈ to C₂₂ alkyl chain, such as N-lauroyl-L-glutamic acid dibutyl amide, manufactured or sold by Ajinomoto under the name GP-1, and mixtures thereof.

The compositions may contain from 1 to 60% of structuring agent present in the fatty phase. Preferably the composition contains from 5 to 55% by weight, better still from 10 to 40%, of structuring agent, which allows total solids contents to be attained in the composition of more than 45%, preferably more than 46%, better still more than 47%, even better more than 48%, and even more than 50%.

One Example of a “Tacky Wax”-Type Structuring Unit Corresponding to the Invention is the Following:

C20-C40 alkyl 12-(12′-hydroxystearyloxy)stearate

Commercial name: Kester wax K82P and Kester wax K 80P from Koster Keunen

Tack=3.38 N.s

Hardness=0.96 MPa

An Example of a Type-2 Structuring Agent Corresponding to the Invention, Consisting of a Semi-Crystalline Polymer in Combination with an Oil, is the Following:

Fatty phase=polybutene(1)/stearyl acrylate-N-vinyl-pyrrolidone (40/60) copolymer (2) with a melting point of 56° C.
(1): Indopol H 100 from AMOCO
(2): Basic polymer with a melting point of 56° C., prepared in accordance with the following procedure:
   A 1 l reactor equipped with a central stirrer with anchor, a condenser and a thermometer is charged with 120 g of cyclohexane, which is heated from the ambient temperature to 80° C. over 45 min. At 80° C., over the course of 2 h, the following mixture C₁ is introduced:
   40 g of cyclohexane+4 g of Triganox 141[2,5-bis(2-ethylhexanoylperoxy)-2,5-dimethylhexane].
   30 min after the beginning of the introduction of the mixture C₁, the mixture C₂ is introduced, over 1 h30 min, this mixture C₂ consisting of:
   190 g of stearyl acrylate+10 g of N-vinyl-pyrrolidone+400 g of cyclohexane.

After the two feeds, the mixture is left for 3 h more at 80° C. and then all of the cyclohexane present in the reaction mixture is distilled off under atmospheric pressure.

This gives the polymer with an active substance content of 100% by weight.

Its weight-average molecular mass Mw is 38 000, expressed in polystyrene equivalents, and its melting point Tf is 56° C. as measured by DSC.

Tack=2.63 N.s

Hardness=5.84 Mpa

According to one second embodiment of the invention, the composition exhibiting the abovementioned features may be obtained by a composition comprising at least one phase comprising at least one aqueous-phase structuring agent which allows the above-defined rheological profile to be obtained.

The aqueous-phase structuring agent may be selected from non-ionic and anionic surfactants which lead to the formation of lamellar phases, amphiphilic polymers leading to the formation of lamellar phases, and associative polymers.

In accordance with the invention, “lamellar phases” are understood to refer to amphiphilic compounds possessing the property of forming, in the presence of water, mesomorphic phases whose organizational state is intermediate between the crystalline state and the liquid state. Among the amphiphiles which give rise to mesomorphic phases, some are able to swell in aqueous solution to form either spherules dispersed in solution or leaflets: these organizations are composed of bimolecular layers.

The lamellar phases are conventionally in a leaflet form, also referred to as bilayers, or in a wound form, also referred to as vesicles, spherules or “onion” phases.

All lamellar phases are characterized under polarized light microscopy by a birefringent structure in the form either of oily streaks or of a Maltese cross, also called a polarization cross.

Examples of Amphiphiles Able to Give Rise to Lamellar Phases:

The amphiphilic molecules may be ionic, preferably anionic, non-ionic, amphoteric or zwitterionic in nature or a combination thereof.

→ Capability to form lamellar phases is possessed by a relatively lipophilic amphiphile (HLB=1-7) or a relatively hydrophilic amphiphile (HLB=8-20) or else a mixture of the 2.
The hydrocarbon surfactants

Possible examples of hydrocarbon surfactants include amphiphilic lipids which are used to obtain vesicles and which have the general formula:
X—Y
in which X represents a hydrophilic group and Y represents a lipophilic group. Amphiphilic lipids may be ionic lipids, for which the group X is ionic, or non-ionic lipids, for which the group X is non-ionic.

Ionic amphiphilic lipids are preferably selected from the group consisting of natural phospho-lipids, modified chemically or enzymatically, or synthetic phospholipids, anionic compounds and gangliosides.

Natural phospholipids include egg lecithin or soya lecithin and sphingomyelin; synthetic phospho-lipids include dipalmitoylphosphatidylcholine; and modified phospholipids include hydrogenated lecithin.

Anionic compounds include those represented by the formula 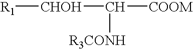
in which
R₁ represents a C₇-C₂₁ alkyl or alkenyl radical;
R₂ represents a saturated or unsaturated C₁-C₃₁ hydrocarbon radical; and
M represents H, Na, K, NH4 or a substituted ammonium ion derived from an amine.
Non-ionic amphiphilic lipids are preferably selected from the group consisting of
(1) linear or branched polyglycerol derivatives of formula 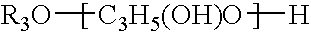
   in which
—C₃H₅(OH)O— is represented by the following structures, taken as a mixture or separately: 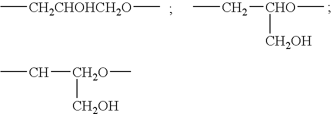
   n is a mean statistical value of between 2 and 6;
R₃ represents
(a) a saturated or unsaturated linear or branched aliphatic chain containing 12 to 30 carbon atoms; or the hydrocarbon radicals of lanolin alcohols;
(b) a residue R′₃CO, in which R′₃ is a C₁₁-C₁₇ linear or branched aliphatic radical;
(c) a residue 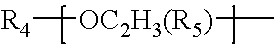
   in which
R₄ may adopt the meaning (a) or (b) given for R₃;
—OC₂H₃(R₅)— is represented by the following structures, taken as a mixture or separately: 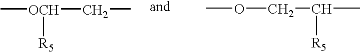
   in which R₅ adopts the meaning (a) given for R₃;
(2) linear or branched polyglycerol ethers containing two fatty chains;
(3) polyoxyethylenated fatty alcohols and polyoxyethylenated sterols and phytosterols;
(4) polyol ethers;
(5) oxyethylenated or non-oxyethylenated polyol esters;
(6) glycolipids of natural or synthetic origin;
(7) hydroxy amides represented by the formula 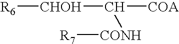
   in which
R₆ denotes a C₇-C₂₁ alkyl or alkenyl radical;
R₇ denotes a saturated or unsaturated C₇-C₃₁ hydrocarbon radical;
COA denotes a group selected from the two following groups:
a residue 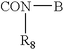
   in which
B is a radical derived from mono- or polyhydroxylated primary or secondary amines; and
R₈ denotes a hydrogen atom or a methyl, ethyl or hydroxyethyl radical; and
a residue —COOZ, where Z represents the residue of a C₃-C₇ polyol.

In the polyglycerol derivatives the saturated linear aliphatic radical R is preferably a lauryl, myristyl, cetyl, stearyl, arachidyl, behenyl or lignoceryl radical or a mixture of these radicals; and the unsaturated aliphatic radical R₃ is advantageously a palmitoleyl, oleyl, linoleyl or arachidonyl radical.

The compounds defined in (3) above are advantageously C₁₂ to C₂₂ alcohols which carry 2 to 20 ethylene oxide (EO) units. The sterol is advantageously cholesterol; it may be substituted by 2 to 20 EO units. Likewise, the phytosterol may be substituted by 2 to 20 mol of EO.

The polyol ethers defined in (4) above are preferably C₂ to C₇ polyol alkyl ethers.

The polyol esters defined in (5) above which are non-ionic and can be used as non-ionic amphiphilic lipids are advantageously cerebrosides.

In accordance with the invention it is possible to incorporate into the lipid phase additives which make it possible to lower the permeability of the vesicles, and/or charged lipids intended to enhance the stability of the vesicles, by preventing their flocculation and their coalescence, and to allow an increase in the degree of encapsulation.

It is also possible in particular to combine, with the amphiphilic lipids which constitute the vesicles, at least one additive selected from the group consisting of:
sterols and their derivatives, for example oxyethylenated derivatives, more particularly cholesterol, acid cholesterol sulphate and its alkali metal salts and acid cholesterol phosphate and its alkali metal salts;
long-chain alcohols and diols;
long-chain amines and their quaternary ammonium derivatives;
dihydroxyalkylamines;
polyoxyethylenated fatty amines;
long-chain amino alcohol esters and their salts and quaternary ammonium derivatives;
phosphoric esters of fatty alcohols, for example dicetyl phosphate and dimyristyl phosphate, in acidic form or in the form of alkali metal salts.

The vesicle dispersions may comprise one or more active compounds having a cosmetic and/or dermo-pharmaceutical activity, which, depending on their solubility characteristics, may have different localizations. If the actives are fat-soluble, they are introduced into the lipid phase constituting the leaflet(s) of the vesicles. If the actives are water-soluble, they are introduced into the encapsulated aqueous phase of the vesicles; if the actives are amphiphilic, they are distributed between the lipid phase and the encapsulated aqueous phase with a partition coefficient which varies depending on the nature of the amphiphilic active and the respective compositions of the lipid phase and of the encapsulated aqueous phase.

Water-soluble actives are, for example, glycerol, sorbitol, erythrulose and antibiotics; fat-soluble actives are, for example, retinoic acid, lipoprotides and steroids.

It is also possible to mention the products having a statistical formula in their hydrophilic portion, for example a polyglycerol ester of formula 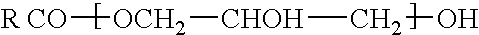
in which n is a statistical value and which may contain various proportions of esters for which n=1, n=2, n=3, n=4, etc.; this is also the case with esters containing a number of alkyl chains in their lipophilic portion, such as cocoates, which contain C₅ to C₁₇ alkyl chains, or isostearates, in which the C₁₇ alkyl chains are a complex mixture of isomeric forms; it is also the case with products consisting of mixtures of mono-, di-, tri- or polyesters of a single polyol. It should be noted that a product containing only a single ester capable of forming vesicles, and impurities of another type, would not be able to be used in accordance with the invention.

Commercial esters which can be used alone in accordance with the invention, since they are in fact mixtures of esters, are, for example, the following:
partial esters of sorbitan (or sorbitol anhydride) and fatty acid, sold under the trade names Span 20, 40, 60 and 80 by ICI;
sorbitan isostearate, sold under the trade. name SI 10 R Nikkol by Nikko;
sorbitan stearate carrying 4 ethylene oxide units, sold under the name Tween 61 by ICI;
polyethylene glycol stearate containing 8 ethylene oxide units, sold under the name MYR J 45 by the company ICI;
polyethylene glycol monostearate of formula:
   OHCH₂(CH₂OCH₂)ₙCH₂OH
   in which n is 4, sold under the name MYS 4 by Nikko;
polyethylene glycol stearate of molecular weight 400, chemical grade or biotechnologically produced grade, sold by Unichema;
diglyceryl stearate carrying 4 ethylene oxide units, sold under the name Hostacerine DGS by Hoechst;
tetraglycerol stearate sold under the name Tetraglyn 1S by Nikko;
diglyceryl isostearate sold by Solvay;
diglyceryl distearate sold under the name Emailex DSG 2 by Nihon;
sucrose mono-, di- and tripalmitostearate, sold under the names F50, F70, F110 and F160 Crodesta by Croda;
mixture of sucrose mono- and di-palmito-stearate, sold under the name Grilloten PSE 141 G by Grillo;
mixture of sucrose stearate and sucrose cocoate, sold under the name Arlatone 2121 by ICI;
methylglucose stearate carrying 20 ethylene oxide units, sold under the name Glucam E 20 distearate by Amerchol.

Mixtures of these various products, which are already mixtures, with one another, or mixtures of these products with pure products, may of course be used.

Ionic amphiphilic lipid(s) in combination with non-ionic amphiphilic lipids according to the invention is(are) preferably taken from the group consisting of:

1) Neutralized anionic lipids, these anionic lipids being preferably selected from
alkali metal salts of dicetyl phosphate and of dimyristyl phosphate, in particular the Na and K salts;
alkali metal salts of cholesterol sulphate, in particular the Na salt;
alkali metal salts of cholesterol phosphate, in particular the Na salt;
mono- and disodium acylglutamates;
the sodium salt of phosphatidic acid;

2) Amphoteric lipids, these amphoteric lipids being preferably phospholipids, in particular pure soya phosphatidylethanolamine;

3) alkylsulphonic derivatives, these derivatives being preferably compounds of formula: 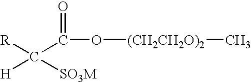
in which R represents the radicals C₁₆H₃₇ and C₁₈H₃₇, taken as a mixture or separately, and M is an alkali metal, preferably sodium.

It is possible conventionally to incorporate, into the lipid phase constituting the lipid membrane of the vesicles, at least one additive whose principal function is to lower the permeability of the vesicles, to prevent their flocculation and their coalescence, and to increase the degree of encapsulation. In accordance with the invention it is possible to add, to the lipid phase, at least one additive selected preferably from the group consisting of
sterols and especially phytosterols and cholesterol,
long-chain alcohols and diols,
long-chain amines and their quaternary ammonium derivatives.
   Silicone surfactants: silicone surfactants which can be used include those described in U.S. Pat. No. 5,364,633 and U.S. Pat. No. 5,411,744. These documents describe the use of silicone surfactants for preparing vesicles.

The silicone surfactant used in accordance with the present invention is preferably a compound of formula (II) 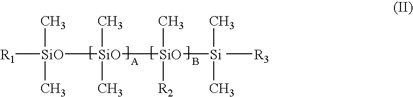
in which
R₁, R₂ and R₃, independently of one another, represent a C₁-C₆ alkyl radical or a radical —(CH₂)x-(OCH₂CH₂)y-(OCH₂CH₂CH₂)z-OR₄, at least one radical R₁, R₂ or R₃ not being an alkyl radical; R₄ being a hydrogen, an alkyl radical or an acyl radical;
A is an integer ranging from 0 to 200;
B is an integer ranging from 0 to 50; on condition that A and B are not simultaneously equal to zero;
x is an integer ranging from 1 to 6;
y is an integer ranging from 1 to 30;
z is an integer ranging from 0 to 5.

In accordance with one preferred embodiment of the invention, in the compound of formula (II), the alkyl radical is a methyl radical, x is an integer ranging from 2 to 6 and y is an integer ranging from 4 to 30.

Examples of silicone surfactants of formula (II) include the compounds of formula (III) 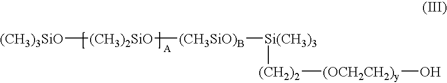
in which A is an integer ranging from 20 to 105, B is an integer ranging from 2 to 10 and y is an integer ranging from 10 to 20.

Examples of silicone surfactants of formula (II) further include the compounds of formula (IV)
HO—(OCH₂CH₂)_{y}—(CH₂)₃—[(CH₃)₂SiO_{A′}—(CH₂)₃—(OCH₂CH₂)_{y}—OH (IV)
in which A′ and y are integers ranging from 10 to 20.

As compounds of the invention it is possible to use those sold by Dow Corning under the names DC 5329, DC 7439-146, DC 2-5695 and Q4-3667. The compounds DC 5329, DC 7439-146 and DC 2-5695 are compounds of formula (III) in which, respectively, A is 22, B is 2 and y is 12; A is 103, B is 10 and y is 12; and A is 27, B is 3 and y is 12.

The compound Q4-3667 is a compound of formula (IV) in which A is 15 and y is 13.

→ Capability to form lamellar phases is possessed for example by the following binary or ternary amphiphile mixtures (CTFA name):

A/cholesterol/casein lipoamino acid, in particular in a 45/45/10 weight ratio (where A is a triglyceryl cetyl ether sold by Chimex under the name Chimexane NL);
B/cholesterol/dicetyl phosphate, in particular in a 60/35/5 weight ratio (where B is a mixture of triglyceryl monocetyl, dicetyl and tricetyl ethers, sold by Chimex under the name Chimexane NT);
Span 40 (from ICI or sorbitan palmitate)/cholesterol/sodium acylglutamate (sold by Ajinomoto under the name HS 11), in particular in a 47.5/47.5/5 weight ratio;
PEG 8 stearate/cholesterol/sodium acylglutamate, in particular with a 47.5/47.5/5 weight ratio (where PEG stearate is the polyethylene glycol containing 8 ethylene oxide units sold by Unichema under the name stearate PEG 400);
PEG 8 stearate/cholesterol/phytanetriol/sodium acylglutamate, in particular with a 47.5/20/27.5/5 weight ratio;
hydrogenated lecithin/phytosterol polyoxy-ethylenated with 5 ethylene oxide units, in particular in a 60/40 weight ratio;
methylglucose distearate polyoxyethylenated with 20 ethylene oxide units/cholesterol/sodium acyl-glutamate, in particular in a 45/45/10 weight ratio (the distearate being, for example, sold under the name Glucam E 20 by Amerchol);
A/cholesterol/dicetyl phosphate, in particular with a 47.5/47.5/5 weight ratio;
diglyceryl distearate (for example that sold by Nihon under the name Emalex DS G2)/cholesterol/sodium acylglutamate, in a 45/45/10 weight ratio;
sucrose mono- and distearate (for example that sold by Grillo under the name Grilloten PSE 141 G)/cholesterol/sodium acylglutamate, in particular in a 45/45/10 weight ratio;
tetraglyceryl tristearate (for example that sold by Nikkol under the name Tetraglyn 3S)/cholesterol/sodium acylglutamate, in particular in a 45/45/10 weight ratio.

Possible examples of vesicles of the second category (delivering the active into the surface layers of the skin) include vesicles obtained from the following lipids:
Sunflower lecithin;
Natipide II (soya lecithin/ethanol/water in a 60/20/20 weight ratio, sold by Nattermann);
C (soya lecithin/cholesterol/propylene glycol in a 40/30/30 weight ratio, sold by Nattermann under the name NAT 50 PG);
D/dimyristyl phosphate in particular in a 95/5 weight ratio (where D is a lauryl polyglyceryl-6-cetearyl glycol ether sold by Chimex under the name Chimexane NS).
→ capability of forming lyotropic lamellar phases is also possessed by amphiphilic polymers such as amphiphilic block copolymers containing asymmetric blocks; for example, those described in the articles below:
amphiphilic diblock copolymers:
   such as, for example, the alcohol ethoxylate diblock copolymer CₙEₙ (C=methylene, E=oxyethylene, the index n describes the number of repeated units) described in the article: Cubic gels and lamellar crystals in concentrated solution of an amphiphilic diblock copolymer. I. W. Hamley et al.—A: Physicochemical and Engineering Aspects 145 (1998), 185-190.
Amphiphilic diblocks or multiblocks, such as, for example, those described in:
   Asymmetric amphiphilic block copolymers in solution: a morphological wonderland, by Neil S. Cameron, Muriel K. Corbierre and A. Eisenberg. Canadian Journal of Chemistry, 1999, p. 2-39.
   Block copolymer assembly to control fluid rheology by S R. Bhatia, A Mourchid, M. Joanicot. Current Opinion in Colloid & Interface Science, 6 (2001) 471-478.
   Thermodynamic size control of block copolymer vesicles in solution, by L. Luo and A. Eisenberg. Langmuir, 2001, 17, 6804-6811.
   Structural polymorphism of amphiphilic copolymers: Six lyotropic liquid crystalline and two solution phases in a poly(oxybutylene)-b-poly(oxyethylene)-water-xylene system. Langmuir, 1997, 13, 23-34.

When at least one of the structuring agents is a lamellar phase it may be in particular based either on anionic surfactant, such as triethanolamine stearate, or on non-ionic surfactant, such as PEG-30 glyceryl stearate (HLB=16.4; Mw=1678).

Associative polymers may be selected from associative polyurethanes, associative acrylic polymers and associative polysaccharides.

Associative polyurethanes are non-ionic block copolymers comprising in the chain both hydrophilic blocks, usually of polyoxyethylenated kind, and hydrophobic blocks, which may be solely aliphatic chain sequences and/or cycloaliphatic and/or aromatic chain sequences.

In particular these polymers contain at least two lipophilic hydrocarbon chains having 6 to 30 carbon atoms, separated by a hydrophilic block, the hydrocarbon chains being either pendant chains or chains of the end of a hydrophilic block. In particular it is possible for one or more pendant chains to be envisaged. Moreover, the polymer may contain a hydrocarbon chain at one end or at the two ends of a hydrophilic block.

The polymers may be in triblock or multiblock form. The hydrophobic blocks may therefore be at each end of the chain (for example: triblock copolymer with a hydrophilic central block) or may be distributed both at the ends and within the chain (multiblock copolymer, for example). The polymers may also be graft polymers or star polymers.

The polymers are preferably triblock copolymers whose hydrophilic block is a polyoxyethylenated chain containing 50 to 1000 oxyethylenated groups. Generally speaking, associative polyurethanes include a urethane linkage between the hydrophilic blocks, which is the origin of the name.

By extension, associative polyurethanes also feature polymers whose hydrophilic blocks are linked to the lipophilic blocks by chemical linkages other than the urethane linkage.

Possible examples of associative polymers which can be used in the invention include the C₁₆-EO₁₂₀-C₁₆ polymer sold by Huls (under the name Serad FX1100, a urethane-functional molecule with a weight-average molecular weight of 1300), EO being an oxyethylene unit. As associative polymer it is also possible to use Rheolate 205, which has a urea function and is sold by Rheox, or else Rheolate 208 or 204. These associative polyurethanes are sold in pure form.

The product DW 1206B from Röhm & Haas, containing a C₂₀ alkyl chain and a urethane linkage, which is sold at a solids content of 20% in water, may also be used.

It is also possible to use solutions or dispersions of these polymers, particularly in water or in an aqueous-alcoholic medium. Possible examples of such polymers include Serad FX1010, Serad FX1035 and Serad 1070, sold by Huls, Rheolate 255, Rheolate 278 and Rheolate 244, sold by Rheox. It is also possible to use the product DW 1206F and DW 1206J, and Acrysol RM 184 or Acrysol 44, from Rohm & Haas, or else Borchigel LW 44 from Borchers.

The polymers which can be used in the invention are in particular those described in the article by G. Fonnum, J. Bakke and Fk. Hansen—Colloid Polym. Sci. 271, 380-389 (1993).

The aqueous-phase structuring agent may be present in the composition in an amount ranging from 0.1 to 15% by weight relative to the total weight of the composition (in particular from 0.1 to 8%), preferably ranging from 0.5 to 12% by weight, (in particular from 0.5% to 5%, or from 0.8% to 3% by weight) and better still ranging from 1% to 8% by weight.

The composition according to the invention may comprise a mixture of a fatty-phase structuring agent and an aqueous-phase structuring agent as defined in the embodiments described above.

The composition according to the invention forms a physiologically acceptable medium.

In the present specification a physio-logically acceptable medium is a non-toxic medium which is compatible with the keratin fibres of human beings, in particular the eyelashes or eyebrows, such as a cosmetic medium, the cosmetic medium being either a hydrophilic or a lipophilic cosmetic medium.

The composition may comprise water or a mixture of water and hydrophilic organic solvent(s), i.e. one or more water-miscible organic solvents, such as alcohols and especially monoalcohols having 2 to 5 carbon atoms, such as ethanol, isopropanol or n-propanol, polyols having 2 to 8 carbon atoms, such as glycerol, diglycerol, propylene glycol, ethylene glycol, 1,3-butylene glycol, sorbitol, pentylene glycol, C₃-C₄ ketones and C₂-C₄ aldehydes.

The water or the mixture of water and hydrophilic organic solvent(s) may be present in the composition according to the invention in an amount ranging in an amount ranging from 0.1% to 90% by weight relative to the total weight of the composition, and preferably from 0.1 to 60% by weight.

According another embodiment, the fatty phase may form a continuous phase of the invention. In particular, the composition of the invention may be anhydrous.

The composition according to the invention is a makeup composition, a makeup base (called “base-coat”), a topcoat composition to be applied over makeup, or a composition for treating or beautifying keratin materials or fibres.

The composition according to the invention is applied more particularly to the eyelashes. Consequently the composition of the invention may be an eyelash coating composition, in particular an eyelash makeup composition, also called mascara, a composition to be applied over an eyelash makeup, also called topcoat, or else an eyelash treatment composition, in particular for treating the eyelashes of human beings or false eyelashes. More especially the composition is a mascara.

The invention also pertains to a cosmetic method for treating or making up keratin fibres, which comprises applying to the said keratin materials the composition as described earlier on.

The invention also relates to a method of coating eyelashes which comprises applying to the eyelashes the composition described above.

The invention further relates to the use of the composition as described earlier on for making up keratin fibres and to the use of this composition for obtaining easy and homogeneous application and a makeup which exhibits an excellent volumizing effect and for obtaining a rapid makeup of keratin fibres.

The composition according to the invention may further comprise an additional wax other than the tackifying wax described hitherto.

The additional wax may be selected for example from beeswax, paraffin waxes, hydrogenated castor oil and silicone waxes.

The waxes (the tackifier wax and/or the additional waxes) present in the composition may be dispersed in particle form in. an aqueous medium. In particular the wax may be present in the form of a wax-in-water emulsion.

The wax or waxes (the tackifier wax and/or the additional wax(es)) present in the composition may also be in the form of an aqueous microdispersion of wax particles. An aqueous microdispersion of wax is an aqueous dispersion of wax particles in which the size of the said wax particles is less than or equal to approximately 1 μm.

Wax microdispersions are stable dispersions of colloidal wax particles, and are described in particular in “Microemulsions Theory and Practice”, L. M Prince Ed., Academic Press (1977), pages 21-32.

These wax microdispersions may in particular be obtained by melting the wax in the presence of a surfactant, and optionally a portion of water, followed by gradual addition of hot water with stirring. The intermediate formation of a water-in-oil emulsion is observed, followed by a phase inversion with final production of an oil-in-water microemulsion. On cooling, a stable microdispersion of solid colloidal wax particles is obtained.

The wax microdispersions may also be obtained by stirring the mixture of wax, surfactant and water using stirring means such as ultrasound, high-pressure homogenizers and turbines.

The particles of the wax microdispersion preferably have average sizes of less than 1 μm (in particular ranging from 0.02 μm to 0.99 μm), preferably less than 0.5 μm (in particular ranging from 0.06 μm to 0.05 μm).

These particles consist essentially of a wax or wax mixture. They may, however, comprise a minor proportion of oily and/or pasty fatty additives, a surfactant and/or a customary fat-soluble active/additive.

The additional wax may be present in the composition according to the invention in an amount ranging from 0.1% to 50% by weight relative to the total weight of the composition, preferably from 0.5% to 30% by weight, and better still from 1% to 20% by weight.

The composition according to the invention may comprise at least one fatty compound which is pastelike at ambient temperature. A pastelike fatty substance in the sense of the invention is a fatty substance having a melting point ranging from 20 to 55° C., preferably from 25 to 45° C., and/or a viscosity at 40° C. ranging from 0.1 to 40 Pa.s (1 to 400 poises), preferably 0.5 to 25 Pa.s measured on the Contraves TV or Rheomat 80, equipped with a rotor rotating at 60 Hz. The skilled worker is able to select the rotor which allows the viscosity to be measured, from the rotors MS-r₃ and MS-r₄, on the basis of his or her general knowledge, so as to be able to carry out measurement on the pastelike test compound.

These fatty substances are preferably hydrocarbon compounds, optionally of polymeric type; they may also be selected from silicone compounds; they may also be in the form of a mixture of hydrocarbon compounds and/or silicone compounds. In the case of a mixture of different pastelike fatty substances it is preferred to use pastelike hydrocarbon compounds (containing primarily carbon atoms and hydrogen atoms and optionally ester groups) in majority proportion.

Among the pastelike compounds which can be used in the composition according to the invention, mention may be made of lanolins and lanolin derivatives such as acetylated lanolins or oxypropylenated lanolins or isopropyl lanolate having a viscosity of 18 to 21 Pa.s, preferably 19 to 20.5 Pa.s, and/or a melting point of 30 to 55° C., and mixtures thereof. It is also possible to use esters of fatty alcohols or acids, particularly those having 20 to 65 carbon atoms (melting point of the order of 20 to 35° C. and/or viscosity at 40° C. ranging from 0.1 to 40 Pa.s) such as triisostearyl or cetyl citrate; arachidyl propionate; polyvinyl laurate; cholesterol esters such as triglycerides of plant origin, such as hydrogenated vegetable oils, viscous polyesters such as poly(12-hydroxystearic) acid, and mixtures thereof.

Mention may also be made of silicone pastelike fatty substances such as polydimethyl-siloxanes (PDMS) having pendant chains of the alkyl or alkoxy type having 8 to 24 carbon atoms, and a melting point of 20-55° C., such as stearyldimethicones, especially those sold by Dow Corning under the trade names DC₂₅03 and DC₂₅514, and mixtures thereof.

The pastelike fatty substance may be present in the composition according to the invention in an amount ranging from 0.01 to 60% by weight relative to the total weight of the composition, preferably ranging from 0.5 to 45% by weight, and better still ranging from 2% to 30% by weight, in the composition.

The composition according to the invention may comprise emulsifying surfactants present in particular in a proportion ranging from 2 to 30% by weight relative to the total weight of the composition, and better still from 5% to 15%. These surfactants may be selected from anionic surfactants and non-ionic surfactants. Reference may be made to Encyclopedia of Chemical Technology, Kirk-Othmer, Volume 22, pp. 333-432, 3rd edition, 1979, Wiley, for the definition of the properties and functions (emulsifier) of the surfactants, in particular to pp. 347-377 of this reference, for anionic and non-ionic surfactants.

The surfactants used preferentially in the composition according to the invention are selected:
from non-ionic surfactants: fatty acids, fatty alcohols, polyethoxylated or polyglycerolated fatty alcohols such as polyethoxylated stearyl or cetylstearyl alcohols, esters of fatty acid and sucrose, esters of alkyl glucose, in particular polyoxyethylenated fatty C₁-C₆ alkyl glucose esters, and mixtures thereof.
from anionic surfactants: C₁₆-C₃₀ fatty acids neutralized with amines, aqueous ammonia or alkali metal salts, and mixtures thereof.

Preference is given to using surfactants which allow an oil-in-water or wax-in-water emulsion to be obtained.

The composition according to the invention may comprise at least one film-forming polymer.

The film-forming polymer may be selected from the group consisting of vinyl polymers, polyurethanes, polyesters, polyamides, polyureas and cellulosic polymers.

The film-forming polymer may be present in the composition according to the invention in a solids content ranging from 0.1% to 60% by weight relative to the total weight of the composition, preferably from 0.5% to 40% by weight, and better still from 1% to 30% by weight.

In the present specification the term “film-forming polymer” refers to a polymer which is capable, by itself or in the presence of an auxiliary film-forming agent, of forming a continuous and adherent film on a support, in particular on keratin materials such as the eyelashes.

The film-forming polymer or polymers which may present in the composition of the invention are different from the “semi-crystalline polymer” as defined earlier on. The film-forming polymer(s) do not preferably comprise crystallizable block(s) or chain(s) . If they do, the crystallizable block(s) or chain(s) represent less than 30% of the total weight of the polymer.

Among the film-forming polymers which may be used in the composition of the present invention, mention may be made of synthetic polymers, of free-radical type or of polycondensate type, and of polymers of natural origin, and mixtures thereof.

A free-radical film-forming polymer is a polymer obtained by polymerizing monomers containing unsaturation, in particular ethylenic unsaturation, each monomer being capable of undergoing homo-polymerization (unlike polycondensates).

The film-forming polymers of free-radical type may in particular be vinyl polymers or copolymers, especially acrylic polymers.

The vinyl film-forming polymers may result from the polymerization of monomers containing ethylenic unsaturation and having at least one acidic group and/or of esters of these acidic monomers and/or of amides of these acidic monomers.

As acid-group-bearing monomer it is possible to use α,β-ethylenic unsaturated carboxylic acids such as acrylic acid, methacrylic acid, crotonic acid, maleic acid and itaconic acid. It is preferred to use (meth)acrylic acid and crotonic acid, and more preferably (meth)acrylic acid.

The esters of acidic monomers are advantageously selected from esters of (meth)acrylic acid (also called (meth)acrylates), in particular alkyl (meth)acrylates, especially C₁-C₃₀, preferably C₁-C₂₀, alkyl (meth)acrylates, aryl (meth)acrylates, especially C₆-C₁₀ aryl (meth)acrylates, and hydroxyalkyl (meth)-acrylates, in particular C₂-C₆ hydroxyalkyl (meth)-acrylates.

Among alkyl (meth)acrylates mention may be made of methyl methacrylate, ethyl methacrylate, butyl methacrylate, isobutyl methacrylate, 2-ethylhexyl meth-acrylate, lauryl methacrylate and cyclohexyl methacrylate.

Among hydroxyalkyl (meth)acrylates mention may be made of hydroxyethyl acrylate, 2-hydroxypropyl acrylate, hydroxyethyl methacrylate, and 2-hydroxy-propyl methacrylate.

Among aryl (meth)acrylates mention may be made of benzyl acrylate and phenyl acrylate.

Particularly preferred esters of (meth)acrylic acid are alkyl (meth)acrylates.

According to the present invention the alkyl group of the esters can be either fluorinated or perfluorinated: in other words, some or all of the hydrogen atoms of the alkyl group are substituted by fluorine atoms.

Possible examples of amides of acidic monomers include (meth)acrylamides, and especially N-alkyl(meth)acrylamides, particularly where the alkyl is C₂-C₁₂. Among N-alkyl(meth)acrylamides mention may be made of N-ethylacrylamide, N-t-butylacrylamide, N-t-octylacrylamide and N-undecylacrylamide.

The vinyl film-forming polymers may also result from the homopolymerization or copolymerization of monomers selected from vinyl esters and styrenic monomers. In particular these monomers can be polymerized with acidic monomers and/or their esters and/or their amides, such as those mentioned hitherto.

Possible examples of vinyl esters include vinyl acetate, vinyl neodecanoate, vinyl pivalate, vinyl benzoate and vinyl t-butyl benzoate.

Styrenic monomers include styrene and alpha-methylstyrene.

Film-forming polycondensates include poly-urethanes, polyesters, polyester amides, polyamides, epoxy ester resins and polyureas.

Polyurethanes may be selected from anionic, cationic, non-ionic or amphoteric polyurethanes, acrylic polyurethanes, polyurethane-polyvinylpyrrolidones, polyester-polyurethanes, polyether-polyurethanes, poly-ureas, polyurea-polyurethanes, and mixtures thereof.

Polyesters may be obtained conventionally by polycondensation of dicarboxylic acids with polyols, especially diols.

The dicarboxylic acid may be aliphatic, alicyclic or aromatic. Possible examples of such acids include oxalic acid, malonic acid, dimethylmalonic acid, succinic acid, glutaric acid, adipic acid, pimelic acid, 2,2-dimethylglutaric acid, azelaic acid, suberic acid, sebacic acid, fumaric acid, maleic acid, itaconic acid, phthalic acid, dodecanedioic acid, 1,3-cyclohexanedicarboxylic acid, 1,4-cyclohexane-dicarboxylic acid, isophthalic acid, terephthalic acid, 2,5-norbornanedicarboxylic acid, diglycolic acid, thiodipropionic acid, 2,5-naphthalenedicarboxylic acid, 2,6-naphthalenedicarboxylic acid. These dicarboxylic acid monomers may be used alone or in a combination of at least two dicarboxylic acid monomers. Among these monomers it is preferred to select phthalic acid, iso-phthalic acid and/or terephthalic acid.

The diol may be selected from aliphatic, alicyclic and aromatic diols. It is preferred to use a diol selected from ethylene glycol, diethylene glycol, triethylene glycol, 1,3-propanediol, cyclohexane-dimethanol and 1,4-butanediol. As other polyols it is possible to use glycerol, pentaerythritol, sorbitol and trimethylolpropane.

The polyester amides may be obtained in a similar way to the polyesters, by a polycondensation of diacids with diamines or amino alcohols. As diamine it is possible to use ethylenediamine, hexamethylene-diamine, meta- or para-phenylenediamine. As an amino alcohol it is possible to use monoethanolamine.

The polyester may further comprise at least one monomer which carries at least one group —SO3M, with M representing a hydrogen atom, an ammonium ion NH4+ or a metal ion, such as, for example, an Na⁺, Li⁺, K⁺, Mg²⁺, Ca²⁺, Cu²⁺, Fe²⁺ or Fe³⁺ ion. It is also possible to use in particular a difunctional aromatic monomer containing such a group —SO₃M.

The aromatic nucleus of the difunctional aromatic monomer additionally carrying a group —SO₃M as described above may be selected, for example, from benzene, naphthalene, anthracene, biphenyl, oxy-biphenyl, sulphonylbiphenyl and methylenebiphenyl nuclei. As examples of a difunctional aromatic monomer further carrying a group —SO₃M, mention may be made of the following: sulphoisophthalic acid, sulpho-terephthalic acid, sulphophthalic acid and 4-sulpho-naphthalene-2,7-dicarboxylic acid.

Preference is given to using copolymers based on isophthalate/sulphoisophthalate, and more particularly copolymers obtained by condensing diethylene glycol, cyclohexanedimethanol, isophthalic acid and/or sulphoisophthalic acid.

The optionally modified polymers of natural origin may be selected from shellac resin, gum sandarac, dammars, elemis, copals, cellulosic polymers, and mixtures thereof.

In accordance with a first embodiment of the composition according to the invention, the film-forming polymer may be a water-soluble polymer and may be present in an aqueous phase of the composition; the polymer is therefore solubilized in the aqueous phase of the composition. Possible examples of water-soluble film-forming polymers include:
proteins such as proteins of plant origin, such as wheat proteins and soya proteins; proteins of animal origin such as keratins, for example keratin hydrolysates and sulphonic keratins;
cellulose polymers such as hydroxyethyl-cellulose, hydroxypropylcellulose, methylcellulose, ethylhydroxyethylcellulose and carboxymethylcellulose, and quaternized cellulose derivatives;
acrylic polymers or copolymers, such as polyacrylates or polymethacrylates;
vinyl polymers, such as polyvinyl-pyrrolidones, copolymers of methyl vinyl ether and malic anhydride, the copolymer of vinyl acetate and crotonic acid, copolymers of vinylpyrrolidone and vinyl acetate; copolymers of vinylpyrrolidone and caprolactam; and polyvinyl alcohol;
polymers of natural origin, optionally modified, such as:
gums arabic, guar gum, xanthan derivatives and karaya gum;
alginates and carragheenans;
glycoaminoglycans and hyaluronic acid and its derivatives;
shellac resin, gum sandarac, dammars, elemis and copals;
deoxyribonucleic acid;
mucopolysaccharides such as chondroitin sulphates,
and mixtures thereof.

In accordance with another variant embodiment of the composition according to the invention, the film-forming polymer may be a polymer which is solubilized in a liquid fatty phase comprising oils or organic solvents such as those described hitherto (in which case the film-forming polymer is referred to as a fat-soluble polymer). A “liquid fatty phase” for the purposes of the invention is a fatty phase which is liquid at ambient temperature (25° C.) and atmospheric pressure (760 mmHg or 105 Pa), which is composed of one or more fatty substances which are liquid at ambient temperature, also called oils, which are generally compatible with one another.

The liquid fatty phase preferably comprises a volatile oil, optionally in a mixture with a non-volatile oil, the oils being selectable from the abovementioned oils.

Possible examples of fat-soluble polymers include vinyl ester copolymers (the vinyl group being connected directly to the oxygen atom of the ester group and the vinyl ester having a saturated, linear or branched hydrocarbon radical of 1 to 19 carbon atoms which is linked to the carbonyl of the ester group) with at least one other monomer which may be a vinyl ester (other than the vinyl ester already present), an α-olefin (having 8 to 28 carbon atoms), an alkyl vinyl ether (in which the alkyl group contains 2 to 18 carbon atoms) or an allyl or methallyl ester (having a saturated, linear or branched hydrocarbon radical of 1 to 19 carbon atoms which is linked to the carbonyl of the ester group).

These copolymers may be crosslinked by means of crosslinkers which can be either of vinyl type or of allyl or methallyl type, such as tetraallyloxyethane, divinylbenzene, divinyl octanedioate, divinyl dodecane-dioate and divinyl octadecanedioate.

As examples of these polymers mention may be made of the following copolymers: vinyl acetate/allyl stearate, vinyl acetate/vinyl laurate, vinyl acetate/vinyl stearate, vinyl acetate/octadecene, vinyl acetate/octadecyl vinyl ether, vinyl propionate/allyl laurate, vinyl propionate/vinyl laurate, vinyl stearate/1-octadecene, vinyl acetate/1-dodecene, vinyl stearate/ethyl vinyl ether, vinyl propionate/cetyl vinyl ether, vinyl stearate/allyl acetate, vinyl 2,2-dimethyloctanoate/vinyl laurate, allyl 2,2-dimethylpentanoate/vinyl laurate, vinyl dimethyl-propionate/vinyl stearate, allyl dimethylpropionate/vinyl stearate, vinyl propionate/vinyl stearate, crosslinked with 0.2% of divinylbenzene, vinyl dimethylpropionate/vinyl laurate, crosslinked with 0.2% of divinylbenzene, vinyl acetate/octadecyl vinyl ether, crosslinked with 0.2% of tetraallyloxyethane, vinyl acetate/allyl stearate, crosslinked with 0.2% of divinylbenzene, vinyl acetate/1-octadecene crosslinked with 0.2% of divinylbenzene, and allyl propionate/allyl stearate crosslinked with 0.2% of divinylbenzene.

Fat-soluble film-forming polymers also include fat-soluble copolymers, and in particular those resulting from the copolymerization of vinyl esters having 9 to 22 carbon atoms or alkyl acrylates or methacrylates, the alkyl radicals having 10 to 20 carbon atoms.

Fat-soluble copolymers of this kind may be selected from copolymers of polyvinyl stearate, polyvinyl stearate crosslinked using divinylbenzene, diallyl ether or diallyl phthalate, copolymers of polystearyl (meth)acrylate, polyvinyl laurate, polylauryl (meth)acrylate, it being possible for these poly(meth)acrylates to be crosslinked using ethylene glycol dimethacrylate or tetraethylene glycol dimethacrylate.

The fat-soluble homopolymers and copolymers defined above are known and are described in particular in the application FR-A-2232303; they can have a weight-average molecular weight ranging from 2000 to 500 000 and preferably from 4000 to 200 000.

As fat-soluble film-forming polymers which can be used in the invention, mention may also be made of polyalkylenes and especially the copolymers of C₂-C₂₀ alkenes, such as polybutene, alkylcelluloses with a linear or branched, saturated or unsaturated C₁ to C₈ alkyl radical such as ethylcellulose and propylcellulose, vinylpyrrolidone (VP) copolymers and specially the copolymers of vinylpyrrolidone and a C₂ to C₄₀ or, better still, C₃ to C₂₀ alkene. Possible examples of VP copolymers which can be used in the invention include VP/vinyl acetate, VP/ethyl ethacrylate, butylated polyvinylpyrrolidone (PVP), VP/ethyl methacrylate/methacrylic acid, VP/eicosene, VP/hexadecadene, VP/triacontene, VP/styrene and VP/acrylic acid/lauryl methacrylate copolymer.

In accordance with another variant embodiment of the composition of the invention, the film-forming polymer may also be present in the composition in the form of particles in dispersion in an aqueous phase or in a non-aqueous solvent phase, known generally under the name latex or pseudolatex. The techniques for preparing these dispersions are well known to the person skilled in the art.

As aqueous dispersions of film-forming polymer it is possible to use the acrylic dispersions sold under the names Neocryl XK-90®, Neocryl A-1070®, Neocryl A-1090®, Neocryl BT-62®, Neocryl A-1079® and Neocryl A-523® by Avecia-Neoresins, Dow Latex 432® by Dow Chemical, Daitosol 5000 AD® by Daito Kasei Kogyo; or else aqueous polyurethane dispersions sold under the names Neorez R-981® and Neorez R-974® by Avecia-Neoresins, Avalure UR-405®, Avalure UR-410®, Avalure UR-425®, Avalure UR-450®, Sancure 875®, Sancure 861®, Sancure 878® and Sancure 2060® by Goodrich, Impranil 85® by the company Bayer, and Aquamere H-1511® by Hydromer; the sulpho polyesters sold under the brand name Eastman AQ® by Eastman Chemical Products, and the vinyl dispersions such as Mexomere PAM and also the acrylic dispersions in isododecane such as Mexomere PAP by Chimex.

The composition according to the invention may comprise a plasticizer which promotes the formation of a film with the film-forming polymer. Such a plasticizer may be selected from all compounds known to the person skilled in the art as being capable of fulfilling the desired function.

The composition according to the invention may further comprise a colorant such as pulverulent colorants, fat-soluble colorants and water-soluble colorants. This colorant may be present in an amount ranging from 0.01% to 30% by weight, relative to the total weight of the composition.

The pulverulent colorants may be selected from pigments, nacres, fat-soluble dyes and water-soluble dyes.

The pigments can be white or coloured, mineral and/or organic, and coated or uncoated. The mineral pigments include titanium dioxide, optionally surface-treated, zirconium oxide, zinc oxide or cerium oxide, and also iron oxides or chromium oxides, manganese violet, ultramarine blue, chromium hydrate and ferric blue. The organic pigments include carbon black, pigments of D & C type, and lakes based on cochineal carmine, barium, strontium, calcium and aluminium.

The nacres may be selected from white nacreous pigments such as mica coated with titanium or with bismuth oxychloride, coloured nacreous pigments such as titanium mica with iron oxides, titanium mica with, in particular, ferric blue or chromium oxide, titanium mica with an organic pigment of the type mentioned above, and nacreous pigments based on bismuth oxychoride.

Fat-soluble dyes are, for example, Sudan Red, D&C Red 17, D&C Green 6, β-carotene, soya oil, Sudan Brown, D&C Yellow 11, D&C Violet 2, D&C Orange 5, quinoline yellow and annatto. Water-soluble dyes are, for example, beetroot juice, methylene blue, the disodium salt of ponceau, the disodium salt of alizarin green, quinoline yellow, the trisodium salt of amaranth, the disodium salt of tartrazine, the monosodium salt of rhodamine, the disodium salt of fuschsin and xanthophyll.

The composition of the invention may further comprise any additive commonly used in cosmetology, such as antioxidants, fillers, preservatives, perfumes, neutralizing agents, thickeners, surfactants, cosmetic or dermatological active agents, plasticizers, coalescents and mixtures thereof.

The person skilled in the art will of course take care to select any complementary additives and/or their amount such that the advantageous properties of the composition according to the invention are not, or not substantially, adversely affected by the intended addition.

The composition according to the invention may be manufactured by the known processes which are generally used within the cosmetics field.

The invention will now be described with reference to the following examples, which are given by way of illustration and are not limitative.

Before going into more detail in these examples we will set out the protocols for measuring the various parameters which allow the present invention to be defined, namely the solids content, the tack value, the hardness and the measurement of the flow profile.

Tack Measurement Protocol

This protocol is valid for determining both the tack of a wax and the tack of the combination of a specific compound with at least one oil, it being possible for the said specific compound to be a semi-crystalline polymer or a fatty-phase rheological agent.

The tack of the fatty-phase structuring agent is measured at 20° C. using a texturometer sold under the name TA-XT2i by Rheo, equipped with an acrylic polymer traveller in the shape of a cone forming an angle of 45° , by measuring the change in the force (compressive force or stretching force) (F) as a function of the time during the following operation:

The traveller is moved at a speed of 0.5 mm/s and then penetrates the structuring agent to a depth of 2 mm. When the traveller has penetrated the wax to the depth of 2 mm, the traveller is held fixed for 1 second (corresponding to the relaxation time) and then withdrawn at a speed of 0.5 mm/s. During the relaxation time the force (compressive force) decreases sharply to reach zero and then, during the withdrawal of the traveller, the force (stretching force) becomes negative before again increasing towards the value 0. The tack corresponds to the integral of the curve of force as a function of time for the portion of the curve corresponding to negative values of the force (stretching force). The value of the tack is expressed in N.s.

To carry out the measurement of the tack of the structuring agent, the structuring agent is melted at a temperature equal to the melting point of the structuring agent +10° C. The melted structuring agent is poured into a container 25 mm in diameter and 20 mm in depth. The structuring agent is recrystallized at ambient temperature (25° C.) for 24 hours such that the surface of the structuring agent is flat and smooth, and then the structuring agent is stored for at least 1 hour at 20° C. before the tack measurement is carried out.

Hardness Measurement Protocol

This protocol is valid for determining both the hardness of a wax and the hardness of the combination of a specific compound with at least one oil, it being possible for the said specific compound to be a semi-crystalline polymer or a fatty-phase rheological agent.

The hardness of the fatty-phase structuring agent is measured at 20° C. using a texturometer sold under the name TA-XT2i by Rheo, equipped with a stainless steel traveller in the shape of a cylinder with a diameter of 2 mm, by measuring the change in the force (compressive force or stretching force) (F) as a function of the time during the following operation:

The traveller is moved at a speed of 0.1 mm/s and then penetrates the structuring agent to a depth of 0.3 mm. When the traveller has penetrated the wax to the depth of 0.3 mm, the traveller is held fixed for 1 second (corresponding to the relaxation time) and then withdrawn at a speed of 0.1 mm/s. During the relaxation time the force (compressive force) decreases sharply to reach zero and then, during the withdrawal of the traveller, the force (stretching force) becomes negative before again increasing towards the value 0. The hardness corresponds to the maximum compressive force measured between the surface of the traveller and the wax at the moment they are contacted. The value of this force is expressed in MPa.

To carry out the measurement of the hardness of the structuring agent, the structuring agent is melted at a temperature equal to the melting point of the structuring agent +20° C. The melted structuring agent is poured into a container 30 mm in diameter and 20 mm in depth. The structuring agent is recrystallized at ambient temperature (25° C.) for 24 hours such that the surface of the structuring agent is flat and smooth, and then the structuring agent is stored for at least 1 hour at 20° C. before the hardness measurement is carried out.

Flow Profile Measurement Protocol

The measurements were carried out on a ThermoRheo RS 75 controlled-stress rheometer, equipped with a thermostated bath. The shear geometry employed possesses a plane/plane symmetry, of diameter 2 cm, with the surface of the planes striated in order to limit the phenomena of sliding on the wall of the planes.

The gap (or sample thickness) is fixed at 300 μm.

The measurements are made at 25° C.±0.5° C.

Measurement Principle

The analysis in flow regime at equilibrium consists in subjecting a sample, starting from a given moment, to an instantaneous stress τ, which is held constant for a time t (waiting time selected such that the permanent regime is reached, t=30 s). Simultaneously the change in the corresponding shear deformation γ is monitored over time and the shear gradient \dot{γ} is recorded when equilibrium is reached.

Protocol

Step 1: Conditioning of the sample at 25° C. for 2 min (without any shearing)

Step 2: Measurement in flow at equilibrium—in controlled stress mode

The application conditions of the shear stress are the following:

Initial shear stress=0.64 Pa

Final shear stress=2000 Pa

Points measured=40

Distribution of points=logarithmic
Increasing stresses are applied to the sample, the stresses being applied only once.
Waiting for a stable value between each stress.
Waiting time between each stress=30 s.
   Analysis of the Results

The results are analysed through the graphical representation of the change in viscosity, written η, as a function of the shear gradient, written \dot{γ} (also called shear rate). This graph shows a first plateau zone, referred to as first newtonian region, which is defined for the low values of \dot{γ} (\dot{γ}≦10⁻³ s¹) in which the viscosity remains constant: this zone represents the viscosity of the product at rest and can be related to the concept of consistency of the mascara.

For higher shear gradients, 10⁻² s⁻¹≦\dot{γ}≦10³ s⁻¹, the viscosity falls and the product begins to move, and then flows, as it undergoes fluidification. Within this shear window there are two possible behaviours:
either flow is homogeneous, in which case the points on the graph are equidistant and the curve is such that all the ratios:

$$\frac{Δ \dot{γ}}{Δτ} \leq 7$$
or flow is inhomogeneous and in this case there is neither continuity nor equidistance between the points on the graph, and the curve is such that some ratios:

$$\frac{Δ \dot{γ}}{Δτ} > 7$$

Solids Content Measurement Protocol

This consists of a measurement of the dry extract of the mascara liquor, which is carried out on a Mettler Toledo HG 53 balance (Halogen Moisture Analyzer).

A sample of mascara (2-3 g) is deposited on an aluminium dish and subjected to a temperature of 120° C. for 60 minutes. The measurement of the dry extract corresponds to the monitoring of the mass of the sample over time. The final solids content is therefore the percentage of the final mass (after 60 min) in relation to the initial mass: DE=(final mass/initial mass)×100, DE corresponding to the solids content defined by a dry solids extract.

The invention will now be described with reference to the following examples, which are given by way of illustration and are not limitative.

### BRIEF DESCRIPTION OF THE DRAWINGS

FIG. 1 represents the flow profile of a mascara-type composition in accordance with the invention, the said profile representing the viscosity (η in Pa.s) as a function of the shear gradient \dot{γ} (in s−1).

FIG. 2 represents the flow profile of a commercial mascara Illusionist® by Estee Lauder, the said profile representing the viscosity (η in Pa.s) as a function of the shear gradient \dot{γ} (in s−1).

FIG. 3 represents the flow profile of a composition in accordance with the invention, the said profile representing the viscosity (η in Pa.s) as a function of the shear gradient \dot{γ} (in s−1).

FIG. 4 represents the flow profile of a commercial mascara Long Optic® from Dior, the said profile representing the viscosity (η in Pa.s) as a function of the shear gradient \dot{γ} (in s−1).

FIG. 5 represents the flow profile of a commercial mascara Effet Faux Cils® from Yves Saint-Laurent, the said profile representing the viscosity (η in Pa.s) as a function of the shear gradient \dot{γ} (in s−1).

### EXAMPLES

### Example 1

Wax-In-Water Emulsion Mascara with Homogeneous and Continuous Flow

Composition

| | |
|---|---|
| Stearic acid | 5.82% |
| Candellila wax | 6% |
| C20-C40 fatty alcohol hydroxystearoyl stearate | 25% |
| (Kester wax K82P) | |
| 2-Amino-2-methylpropane-1,3-diol | 0.5% |
| Black iron oxide | 5.45% |
| Silica | 3% |
| Hydroxyethylcellulose | 0.91% |
| Gum arabic | 3.45% |
| Triethanolamine | 2.4% |
| Antifoam | qs |
| Preservative | qs |
| Water | qs |

Solids content measured = 53%

For this example,

$$\frac{Δ \dot{γ}}{Δτ}$$

varies from 4.5×10−4 to 4.3 within the shear gradient range between 1×10⁻² s⁻¹ and 1×10³ s⁻¹.

The mascara of this example is capable of withstanding high shears of the order of 1000 s−1.

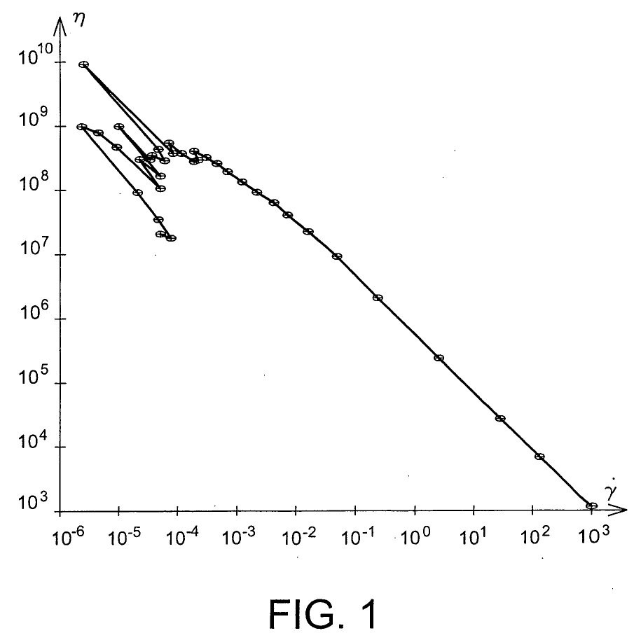

FIG. 1 represents the flow profile of the said composition, representing the viscosity of the composition as a function of the shear gradient.

It confirms that the flow is continuous and homogeneous and that this composition is able to undergo stress up to a shear gradient of 10⁺³ s⁻¹.

A makeup product is obtained which loads the eyelashes and is smooth and homogeneous.

### Comparative Example 1

The composition subjected to the abovementioned rheological profile measurement protocol is a commercial composition, Illusionist® from Estee Lauder.

Solids content measured=46.8%

For this composition,

$$\frac{Δ \dot{γ}}{Δτ}$$

varies from 4.35×10^{31 4} to 8.5 within the shear gradient range between 1×10⁻² s⁻¹ and 1×10³ s⁻¹. Insofar as this ratio exceeds 7, the flow is therefore discontinuous and inhomogeneous.
Illusionist mascara is incapable of withstanding shears of 1000 s−1 since \dot{γ} max=400 s−1.

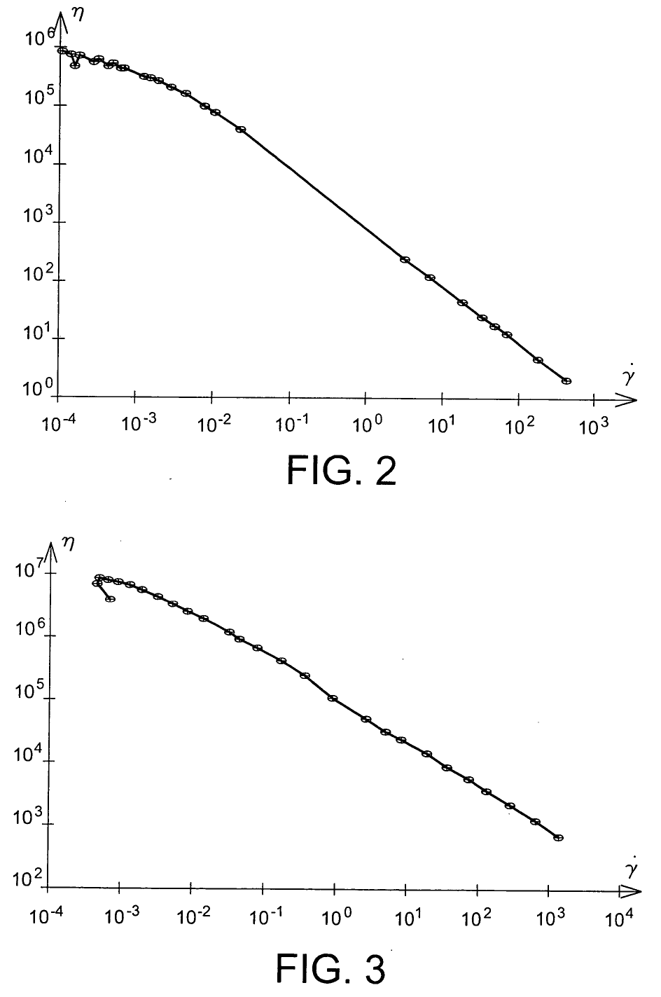

FIG. 2 confirms for this type of mascara a discontinuous and non-homogeneous flow.

This mascara imparts a natural makeup (fairly thin) to the eyelashes and forms a granular deposit.

### Example 2

Wax-in-Water Emulsion Mascara with Non-Ionic Surfactant (Corresponding to a Lamellar Phase) Exclusively and Exhibiting Homogeneous and Continuous Flow

Composition:

| | |
|---|---|
| Beeswax | 4.63% |
| Hydrogenated jojoba oil | 0.11% |
| Carnauba wax | 3.41% |
| Rice bran wax | 0.11% |
| Paraffin wax | 14.64% |
| Candelilla wax | 0.32% |
| Black iron oxide | 7.42% |
| Hydroxyethylcellulose | 0.94% |
| Gum arabic | 3.59% |
| Glyceryl monostearate 30EO (= Tagat S) | 2.54% |
| Mixture of polydimethylsiloxane containing | 8.5% |
| alpha, omega-hydroxyl groups and | |
| cyclopentadimethylsiloxane (15/85) | |
| Stearyl alcohol 2 EO | 1.5% |
| Stearyl alcohol 20 EO | 1% |
| Antifoam | qs |
| Preservative | qs |
| Water | qs |

Solids content measured = 45.24%

For this example,

$$\frac{Δ \dot{γ}}{Δτ}$$

varies from 1.5×10⁻³ to 3.9 within the shear gradient range between 1×10⁻² s−1 and 1×10³ s−1. This mascara therefore exhibits continuous and homogeneous flow.

This mascara of Example 1 is capable of withstanding high shears of the order of 1000 s−1, and this is confirmed by FIG. 3.

This mascara imparts good volumizing properties (thick makeup on the lashes) and forms a smooth and homogeneous deposit.

### Comparative Example 2

### Long Optic from Dior

Solids content=42.5%, measured

$$\frac{Δ \dot{γ}}{Δτ}$$

varies from 1.2×10⁻³ to 30.8 within the shear gradient range between 1×10⁻² s⁻¹ and 1×10³ s⁻¹.

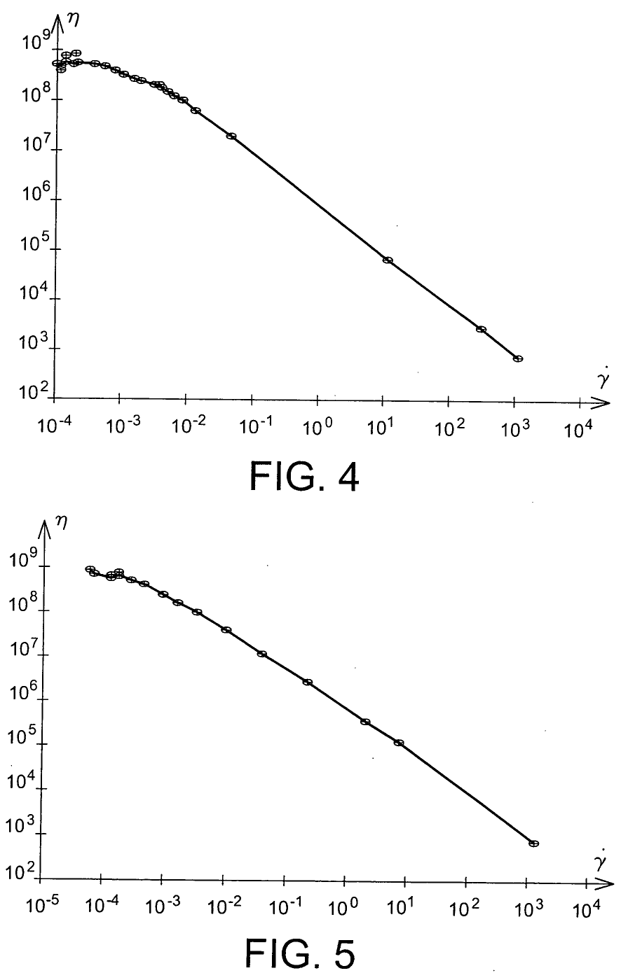

This mascara does not fall within the scope of our invention, since the flow is inhomogeneous above 10⁻² s⁻¹, and this is confirmed by FIG. 4.

This mascara imparts a natural makeup to the eyelashes (relatively thin makeup) and forms an unsmooth deposit.

### Comparative Example 3

### Effet Faux Cils from from Yves Saint Laurent

Solids content=52.7%, measured

$$\frac{Δ \dot{γ}}{Δτ}$$

varies from 1.8×10⁻³ s⁻¹ to 7.5 within the shear gradient range between 1×10⁻² s⁻¹ and 1×10³ s⁻¹.

The EFC mascara does not fall within the scope of our invention, since the flow is inhomogeneous above 10 s−1, and this is confirmed by FIG. 5.

This mascara imparts a makeup effect with loading of the lashes, but forms an inhomogeneous deposit.

1-31. (canceled)

32. A composition comprising a solids content of more than 45% of the total weight of the composition and having a rheological profile such that, for a shear gradient range from 10⁻² to 10³ s⁻¹, the ratio of the change in shear gradient to the change in applied shear stress
$$\frac{Δ \dot{γ}}{Δτ}$$
is not more than 7; wherein the composition is capable of undergoing shear stresses over the entire breadth of said shear gradient range without breaking up.

33. The composition according to claim 32, wherein the ratio
$$\frac{Δ \dot{γ}}{Δτ}$$
is less than 5.

34. The composition according to claim 32, wherein the solids content is greater than 50% of the total weight of the composition.

35. The composition according to claim 32, further comprising at least one fatty phase, said fatty phase comprising at least one fatty-phase structuring agent.

36. The composition according to claim 35, wherein the at least one structuring agent is selected from the group consisting of tackifying waxes, a combination of at least one specific compound with at least one oil, and mixtures thereof.

37. The composition according to claim 36, comprising a tackifying wax having the following features:
a tack ≦0.7 N.s,
a hardness ≧3.5 MPa,.

38. The composition according to claim 35, wherein said structuring agent consists of the combination of at least one specific compound with at least one oil.

39. The composition according to claim 38, wherein the specific compound is selected from the group consisting of semi-crystalline polymers, fatty-phase rheological agents and mixtures thereof.

40. The composition according to claim 38, wherein the at least one oil is selected from the group consisting of volatile and non-volatile hydrocarbon oils, silicone oils and/or fluoro oils, and mixtures thereof.

41. The composition according to claim 38, comprising an oil having a molecular mass greater than or equal to 250 g/mol.

42. The composition according to claim 38, wherein the combination has:
a tack ≦0.1 N.s,
a hardness ≧30 MPa.

43. The composition according to claim 38, wherein the combination comprises a semi-crystalline polymer and exhibits a tack from 1 to 5 N.s and a hardness value of from 0.1 to 20 MPa.

44. The composition according to claim 35, wherein the structuring agent represents from 0.1 to 60% of the total weight of the composition.

45. The composition according to claim 32, further comprising at least one aqueous phase comprising at least one aqueous-phase structuring agent.

46. The composition according to claim 45, wherein said aqueous-phase structuring agent is selected from the group consisting of non-ionic and anionic surfactants leading to the formation of lamellar phases, amphiphilic polymers leading to the formation of lamellar phases, associative polymers, and mixtures thereof.

47. The composition according to claim 32, further comprising water and, optionally, one or more hydrophilic organic solvents.

48. The composition according to claim 47, comprising at least one hydrophilic organic solvent selected from the group consisting of monoalcohols having from 2 to 5 carbon atoms, polyols having from 2 to 8 carbon atoms, C₃-C₄ ketones, C₂-C₄ aldehydes, and mixtures thereof.

49. The composition according to claim 47, wherein the water or the mixture of water and hydrophilic organic solvent(s) is present in an amount of 0.1% to 90% by weight, relative to the total weight of the composition.

50. The composition according to claim 32, further comprising a film-forming polymer.

51. The composition according to claim 50, wherein the film-forming polymer is selected from the group consisting of vinyl polymers, polyurethanes, polyesters, polyamides, polyureas, cellulosic polymers and mixtures thereof.

52. The composition according to claim 50, wherein the film-forming polymer is present at a polymer solids content ranging from 0.1% to 60% by weight relative to the total weight of the composition.

53. The composition according to claim 32, further comprising a colorant.

54. The composition according to claim 53, wherein the colorant is selected from the group consisting of pigments, nacres, fat-soluble dyes, water-soluble dyes, and mixtures thereof.

55. The composition according to claim 53, wherein the colorant is present in an amount ranging from 0.01% to 30% by weight relative to the total weight of the composition.

56. The composition according to claim 32, further comprising an additive selected from the group consisting of antioxidants, fillers, preservatives, perfumes, neutralizing agents, thickeners, surfactants, cosmetic or dermatological active agents, plasticizers, coalescents and mixtures thereof.

57. The composition according to claim 32, wherein the composition is a makeup base, a top-coat composition to be applied over makeup, or a composition for treating or beautifying keratin materials or fibers.

58. The composition according to claim 32, wherein the composition is an eyelash coating composition, a composition to be applied over an eyelash makeup, or an eyelash treatment composition for treating the eyelashes of human beings or false eyelashes.

59. The composition according to claim 32, wherein the composition is a mascara.

60. A method for treating or making up keratin fibers, which comprises applying to the keratin fibers the composition according to claim 32.

61. A method of coating eyelashes, which comprises applying to the eyelashes the composition according to claim 32.

62. A method for making up and/or volumizing keratin fibers comprising applying thereto the composition of claim 32.
